(19) United States
(12) Patent Application Publication
(10) Pub. No.: US 2025/0263746 A1
(43) Pub. Date: Aug. 21, 2025

(54) METHOD FOR PRODUCING RECOMBINANT AAV PARTICLES

(71) Applicant: Hoffmann-La Roche Inc., Little Falls, NJ (US)

(72) Inventors: Christoph FEISTL, Penzberg (DE); Marc POMPIATI, Penzberg (DE); Monika POPP, Penzberg (DE); Johanna Alina SCHNEIDER, Penzberg (DE)

(73) Assignee: Hoffmann-La Roche Inc., Little Falls, NJ (US)

(21) Appl. No.: 19/019,201

(22) Filed: Jan. 13, 2025

Related U.S. Application Data
(63) Continuation of application No. PCT/EP2023/069338, filed on Jul. 12, 2023.

(30) Foreign Application Priority Data
Jul. 14, 2022 (EP) 22184964.9

(51) Int. Cl. C12N 15/86

(52) U.S. Cl. CPC C12N 15/86; C12N 2750/14143; C12N 2750/14151

(57) ABSTRACT

Herein is reported a method for lysing recombinant AAV particle producing mammalian cells comprising the step of bringing a mammalian cell cultivation broth in contact with an alkyl polyglucoside detergent, preferably Triton CG 110, and thereby lysing recombinant AAV particle producing mammalian cells and releasing the produced recombinant AAV particles, wherein the mammalian cell cultivation broth comprises cultivated recombinant AAV particle producing mammalian cells and the cultivation medium used for the cultivation of said recombinant AAV particle producing mammalian cells (spent medium).

### CROSS-REFERENCE TO RELATED APPLICATIONS

This application is a continuation of PCT Application No. PCT/EP2023/069338 filed Jul. 12, 2023, which claims priority to European Application No. 22184964.9 filed Jul. 14, 2022, each of which is incorporated by reference in its entirety.

### FIELD OF INVENTION

The current invention is in the field of gene therapy. More precisely herein is reported a method for the release of recombinant AAV particles from the producing cells, wherein the cells are lysed using Triton CG 110.

### BACKGROUND

Gene therapy refers broadly to the therapeutic administration of genetic material to modify gene expression of living cells and thereby alter their biological properties. After decades of research, gene therapies have progressed to the market and are expected to become increasingly important. In general, gene therapy can be divided into either in vivo or ex vivo approaches.

Today, most in vivo therapies rely on DNA delivery with recombinant adeno-associated viral (rAAV) vectors. An AAV is a small, naturally occurring, non-pathogenic parvovirus, which is composed of a non-enveloped icosahedral capsid. It contains a linear, single stranded DNA genome of approximately 4.7 kb. The genome of wild-type AAV vectors carries two genes, rep and cap, which are flanked by inverted terminal repeats (ITRs). ITRs are necessary in cis for viral replication and packaging. The rep gene encodes for four different proteins, whose expression is driven by two alternative promoters, P5 and P19. Additionally different forms are generated by alternative splicing. The Rep proteins have multiple functions, such as, e.g., DNA binding, endonuclease and helicase activity. They play a role in gene regulation, site-specific integration, excision, replication and packaging. The cap gene codes for three capsid proteins and one assembly-activating protein. Differential expression of these proteins is accomplished by alternative splicing and alternative start codon usage and is driven by a single promoter, P40, which is located in the coding region of the rep gene.

In engineered, therapeutic rAAV vectors, the viral genes are replaced with a transgene expression cassette, which remains flanked by the viral ITRs, but encodes a gene of interest under the control of a promoter of choice. Unlike the wild-type virus, the engineered rAAV vector does not undergo site-specific integration into the host genome, remaining instead predominantly episomal in the nucleus of transduced cells.

An AAV is not replication competent by itself but requires the function of helper genes. These are provided in nature by co-infected helper viruses, such as, e.g., adenovirus or herpes simplex virus. For instance, five adenoviral genes, i.e. E1A, E1B, E2A, E4 and VA, are known to be essential for AAV replication. In contrast to the other helper genes, which code for proteins, VA is a small RNA gene.

For the production of rAAV vectors, DNA carrying the transgene flanked by ITRs is introduced into a packaging host cell line, which also comprises rep and cap genes as well as the required helper genes. There are many ways of introducing these three groups of DNA elements into cells and ways of combining them on different DNA plasmids (see, e.g., Robert, M. A., et al. Biotechnol. J. 12 (2017) 1600193).

Two general production methods are widely used. In the triple transfection method, a plasmid comprising rep/cap and a plasmid comprising the rAAV-transgene are transiently co-transfected with an adenovirus helper plasmid carrying the required adenoviral helper genes. The process can be performed using CHO or HEK cells. Alternatively, rep/cap and viral helper genes can be combined on one larger plasmid (dual transfection method). The second method encompasses the infection of insect cells (Sf9) with two baculoviruses, one carrying the rAAV genome and the other carrying rep and cap. In this system, helper functions are provided by the baculovirus plasmid itself. In the same way, herpes simplex virus is used in combination with HEK293 cells or BHK cells. More recently Mietzsch et al. (Hum. Gene Ther. 25 (2014) 212-222; Hum. Gene Ther. Methods 28 (2017) 15-22) engineered Sf9 cells with rep and cap stably integrated into the genome. With these cells, a single baculovirus carrying the rAAV transgene is sufficient to produce rAAV vectors. Clark et al. (Hum. Gene Ther. 6 (1995) 1329-1341) generated a HeLa cell line with rep/cap genes and a rAAV transgene integrated in its genome. By transfecting the cells with wild-type adenovirus, rAAV vector production is induced and mixed stocks of rAAV vectors and adenovirus are produced.

Arvind Srivastava et al. reported about the manufacturing challenges and rational formulation development for AAV viral vectors (J. Pharm Sci. 110 (2021) 2609-2624).

Mafalda Moleirinho et al. reported about clinical-grade oncolytic adenovirus purification using Polysorbate 20 as an alternative for cell lysis (Curr. Gene Ther. 18 (2018) 1-9). WO 2022/003565 reported about a detergent and method for purifying a biotherapeutic.

### SUMMARY OF THE INVENTION

The current invention is based at least in part on the finding that the recovery, i.e. yield, of recombinant AAV particles (both capsid-based yield as well as genome-based yield) in an AAV affinity chromatography is influenced/is depending on the detergent used for lysing the cells that produced the recombinant AAV particle prior to the AAV affinity chromatography.

The current invention is further based at least in part on the finding that the ratio of full recombinant AAV particles to empty recombinant AAV particles, which are obtained in an AAV affinity chromatography, is influenced/is depending on the detergent used for lysing the cells that produced the recombinant AAV particle prior to the AAV affinity chromatography.

It has been found that both (capsid-based as well as genomic) yield as well as the ratio of full to empty recombinant AAV particles can be increased in an AAV affinity chromatography by using an alkyl polyglucoside detergent for lysing the cells that produced the recombinant AAV particle prior to the AAV affinity chromatography step.

The prior art does not suggest to use an alkyl polyglucoside (APG) for lysis of AAV producing cells, let alone for solving the problem of providing a method for increasing the yield and the ratio of full to empty recombinant AAV particles. Thus, the current invention encompasses the following embodiments:
1. A method for lysing recombinant AAV particle producing mammalian cells comprising the following step:
   bringing a mammalian cell cultivation broth in contact with an alkyl polyglucoside detergent,
   and thereby lysing recombinant AAV particle producing mammalian cells,
   wherein the mammalian cell cultivation broth comprises cultivated recombinant AAV particle producing mammalian cells and the cultivation medium used for the cultivation of said recombinant AAV particle producing mammalian cells (spent medium).
2. A method for releasing recombinant AAV particles from recombinant AAV particle producing mammalian cells comprising the following step:
   bringing a mammalian cell cultivation broth in contact with an alkyl polyglucoside detergent,
   and thereby lysing recombinant AAV particle producing mammalian cells,
   wherein the mammalian cell cultivation broth comprises cultivated recombinant AAV particle producing mammalian cells and the cultivation medium used for the cultivation of said recombinant AAV particle producing mammalian cells (spent medium).
3. A method for purifying recombinant AAV particles comprising the following steps:
   bringing a mammalian cell cultivation broth in contact with an alkyl polyglucoside detergent,
   removing dell debris from the mixture, and
   purifying the recombinant AAV particle with an AAV affinity chromatography,
   and thereby purifying recombinant AAV particles,
   wherein the mammalian cell cultivation broth comprises cultivated recombinant AAV particle producing mammalian cells and the cultivation medium used for the cultivation of said recombinant AAV particle producing mammalian cells (spent medium).
4. A method for purifying recombinant AAV particles comprising the following step:
   releasing recombinant AAV particles from the producing mammalian cells by contacting the respective mammalian cell cultivation broth with an alkyl polyglucoside detergent, and
   purifying the recombinant AAV particles with an AAV affinity chromatography
   thereby purifying the recombinant AAV particles,
   wherein the mammalian cell cultivation broth comprises cultivated recombinant AAV particle producing mammalian cells and the cultivation medium used for the cultivation of said recombinant AAV particle producing mammalian cells (spent medium).
5. A method for producing recombinant AAV particles comprising a nucleic acid that encodes a protein of interest or is transcribed into a transcript of interest, wherein the method comprising the following steps:
   (i) providing one or more plasmids comprising nucleic acids encoding AAV packaging proteins and/or nucleic acids encoding helper proteins;
   (ii) providing a plasmid comprising a nucleic acid that encodes a protein of interest or is transcribed into a transcript of interest that is interspaced between AAV ITRs;
   (iii) contacting one or more mammalian cells with the provided plasmids and either further adding a transfection reagent and optionally incubating the plasmid/transfection reagent/cell mixture; or providing a physical means, such as an electric current, to introduce the nucleic acid into the cells;
   (iv) cultivating the transfected cells;
   (v) harvesting the cultivated cells and culture medium to produce a mammalian cell cultivation broth;
   (vi) lysing the cells by bringing the mammalian cell cultivation broth in contact with an alkyl polyglucoside detergent to produce a mammalian cell cultivation broth lysate; and
   (vii) optionally isolating recombinant AAV particles from the cultivation broth lysate with an AAV affinity chromatography;
   thereby producing a recombinant AAV particle comprising a nucleic acid that encodes a protein of interest or is transcribed into a transcript of interest.
6. A method for producing recombinant AAV particles comprising a nucleic acid that encodes a protein of interest or is transcribed into a transcript of interest, wherein the method comprising the following steps:
   (i) providing one or more plasmids comprising nucleic acids encoding AAV packaging proteins and/or nucleic acids encoding helper proteins;
   (ii) providing a plasmid comprising a nucleic acid that encodes a protein of interest or is transcribed into a transcript of interest;
   (iii) either
      (a) generating a stable transfected cell by contacting one or more mammalian cells with the provided plasmids of (i) by either further adding a transfection reagent and optionally incubating the plasmid/transfection reagent/cell mixture or providing a physical means, such as an electric current, to introduce the nucleic acid into the cells; selecting a first stably transfected cell; contacting the selected first stably transfected cell with the provided plasmid of (ii) and either further adding a transfection reagent and optionally incubating the plasmid/transfection reagent/cell mixture or providing a physical means, such as an electric current, to introduce the nucleic acid into the cells; or
      (b) generating a transient transfected cell by contacting one or more mammalian cells with the provided plasmids of (i) and (ii) and either further adding a transfection reagent and optionally incubating the plasmid/transfection reagent/cell mixture, or providing a physical means, such as an electric current, to introduce the nucleic acid into the cells;
      thereby generating a transfected cell;
   (iv) cultivating the transfected cell of (iii);
   (v) harvesting the cultivated cells and the culture medium to produce a mammalian cell cultivation broth;
   (vi) lysing the cells by bringing the mammalian cell cultivation broth in contact with an alkyl polyglucoside detergent to produce a mammalian cell cultivation broth lysate; and
   (vii) optionally isolating recombinant AAV particles from the mammalian cell cultivation broth lysate with an AAV affinity chromatography;
   thereby producing recombinant AAV particles comprising a nucleic acid that encodes a protein of interest or is transcribed into a transcript of interest.
7. A method for purifying recombinant AAV particles comprising the steps of:
   a) harvesting cultivated recombinant AAV particle producing mammalian cells and cell culture supernatant comprising rAAV particles to produce a mammalian cell cultivation broth;
   b) optionally concentrating the harvest produced in step (a) to produce a concentrated mammalian cell cultivation broth;
   c) lysing the mammalian cells contained in the mammalian cell cultivation broth produced in step (a) or the concentrated mammalian cell cultivation broth produced in step (b) by bringing the broth in contact with an alkyl polyglucoside detergent to produce a mammalian cell cultivation broth lysate;
   d) treating the lysate produced in step (c) to reduce contaminating nucleic acid in the lysate thereby producing a nucleic acid reduced lysate;
   e) optionally filtering the nucleic acid reduced lysate produced in step (d) to produce a clarified lysate, and optionally diluting the clarified lysate to produce a diluted clarified lysate;
   f) subjecting the nucleic acid reduced lysate obtained in step (d), or clarified lysate or diluted clarified lysate produced in step (e) to an AAV affinity column chromatography to produce a column eluate comprising recombinant AAV particles, thereby separating rAAV particles from protein impurities or other production/process related impurities, and optionally concentrating the column eluate to produce a concentrated column eluate;
   thereby purifying recombinant AAV particles.
8. The method according to embodiment 7 further comprising the following steps:
   g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a diluted second column eluate;
   h) optionally subjecting the second column eluate or the diluted second column eluate produced in step (g) to an anion exchange chromatography to produce a third column eluate comprising rAAV particles, thereby separating rAAV particles from protein impurities or other production/process related impurities, and optionally diluting the third column eluate to produce a diluted third column eluate; and
   i) filtering the second column eluate or the diluted second column eluate produced in step (g), or filtering the third column eluate or the concentrated third column eluate produced in step (h),
   and thereby purifying recombinant AAV particles.
9. The method according to embodiment 7 further comprising the following steps
   g) subjecting the column eluate or the diluted column eluate produced in step (f) to a cation exchange column chromatography to produce a second column eluate comprising rAAV particles, thereby separating rAAV particles from protein impurities or other production/process related impurities, and optionally diluting the column eluate to produce a diluted second column eluate;
   h) subjecting the column eluate or the diluted column eluate produced in step (g) to an anion exchange chromatography to produce a third column eluate comprising rAAV particles, thereby separating rAAV particles from protein impurities or production/process related impurities, and optionally concentrating the third column eluate to produce a concentrated third column eluate,
   and thereby purifying recombinant AAV particles.
10. The method according to embodiment 7 further comprising the following steps:
   g) subjecting the column eluate or the diluted column eluate produced in step (f) to an anion exchange chromatography to produce a second column eluate comprising rAAV particles, thereby separating rAAV particles from protein impurities or production/process related impurities, and optionally concentrating the second column eluate to produce a concentrated second column eluate;
   h) subjecting the column eluate or the diluted column eluate produced in step (g) to a cation exchange column chromatography to produce a third column eluate comprising rAAV particles, thereby separating rAAV particles from protein impurities or other production/process related impurities, and optionally concentrating the third column eluate to produce a concentrated third column eluate,
   and thereby purifying recombinant AAV particles.
11. Use of an alkyl polyglucoside detergent for lysing recombinant AAV particle producing mammalian cells for increasing the yield of recombinant AAV particles obtained in a subsequent AAV affinity chromatography.
12. Use of an alkyl polyglucoside detergent for lysing recombinant AAV particle producing mammalian cells for increasing the ratio of full recombinant AAV particles to empty recombinant AAV particles (in eluate fractions) obtained in a subsequent AAV affinity chromatography.
13. Use of an alkyl polyglucoside detergent for increasing the yield of recombinant AAV particles, wherein AAV particle producing mammalian cells are lysed with the alkyl glucoside detergent and wherein the recombinant AAV particles obtained in a subsequent AAV affinity chromatography.
14. Use of an alkyl polyglucoside detergent for increasing the yield of recombinant AAV particles, wherein recombinant AAV particle producing mammalian cells are lysed with an alkyl polyglucoside detergent and wherein the yield is determined after a subsequent AAV affinity chromatography step.
15. Use of an alkyl polyglucoside detergent for increasing the ratio of full recombinant AAV particles to empty recombinant AAV particles (in eluate fractions) obtained in a subsequent AAV affinity chromatography, wherein recombinant AAV particle producing mammalian cells are lysed with an alkyl polyglucoside detergent prior to the AAV affinity chromatography.
16. The method and use according to any one of the preceding embodiments, wherein the alkyl polyglucoside detergent is a mixture of 58.0-62.0 (w/v) % D-glucopyranose, oligomeric, decyl octyl glycoside and 38.0-42.0 (w/v) % water.
17. The method and use according to any one of the preceding embodiments, wherein the alkyl polyglucoside detergent has the CAS number 68515-73-1.
18. The method and use according to any one of the preceding embodiments, wherein the mammalian cell cultivation broth is a crude mammalian cell cultivation broth.
19. The method and use according to any one of the preceding embodiments, wherein the bringing in contact or contacting with the alkyl polyglucoside detergent is an incubating with the alkyl polyglucoside detergent for a defined time at a defined concentration of the alkyl polyglucoside detergent.
20. The method and use according to any one of the preceding embodiments, wherein the alkyl polyglucoside detergent is in a solution.
21. The method and use according to any one of the preceding embodiments, wherein the mammalian cell cultivation broth is combined with 2.5% to 20% of its volume (2.5% (v/v) to 20% (v/v)) with a solution comprising an alkyl polyglucoside detergent.
22. The method and use according to any one of the preceding embodiments, wherein the mammalian cell cultivation broth is combined with 5% to 10% of its volume (5% (v/v) to 10% (v/v)) with a solution comprising an alkyl polyglucoside detergent.
23. The method and use according to any one of embodiments 20 to 22, wherein the solution comprising the alkyl polyglucoside detergent comprises the alkyl polyglucoside detergent at a concentration of 5% to 20%.
24. The method and use according to any one of embodiments 20 to 22, wherein the solution comprising the alkyl polyglucoside detergent comprises the alkyl polyglucoside detergent at a concentration of 7.5% to 15%.
25. The method and use according to any one of embodiments 20 to 22, wherein the solution comprising the alkyl polyglucoside detergent preferably comprises the alkyl polyglucoside detergent at a concentration of about 10%.
26. The method and use according to any one of the preceding embodiments, wherein bringing into contact or the contacting, respectively, with the alkyl polyglucoside detergent is for 30 min. to 90 min.
27. The method and use according to any one of the preceding embodiments, wherein bringing into contact or the contacting, respectively, with the alkyl polyglucoside detergent is for 45 min. to 75 min.
28. The method and use according to any one of the preceding embodiments, wherein bringing into contact or the contacting, respectively, with the alkyl polyglucoside detergent preferably is for about 60 min.
29. The method and use according to any one of the preceding embodiments, wherein bringing into contact or the contacting, respectively, with the alkyl polyglucoside detergent is at a temperature of 25° C. to 45° C.
30. The method and use according to any one of the preceding embodiments, wherein bringing into contact or the contacting, respectively, with the alkyl polyglucoside detergent is at a temperature of 28° C. to 42° C.
31. The method and use according to any one of the preceding embodiments, wherein bringing into contact or the contacting, respectively, with the alkyl polyglucoside detergent is at a temperature of 32° C. to 40° C.
32. The method and use according to any one of the preceding embodiments, wherein bringing into contact or the contacting, respectively, with the alkyl polyglucoside detergent preferably is at a temperature of about 37° C.
33. The method and use according to any one of the preceding embodiments, wherein bringing into contact or the contacting, respectively, with the alkyl polyglucoside detergent is with stirring or with shaking.
34. The method and use according to any one of the preceding embodiments, wherein bringing into contact or the contacting, respectively, with the alkyl polyglucoside detergent is without aeration.
35. The method and use according to any one of the preceding embodiments, wherein bringing into contact or the contacting, respectively, with the alkyl polyglucoside detergent is without pH adjustment during the bringing into contact or contacting.
36. The method and use according to any one of the preceding embodiments, wherein after the bringing into contact or the contacting, respectively, with the alkyl polyglucoside detergent diatomaceous earth is added and the mixture is incubated for 5 to 20 min.
37. The method and use according to any one of the preceding embodiments, wherein after the bringing into contact or the contacting, respectively, with the alkyl polyglucoside detergent or the diatomaceous earth the mixture is centrifuged or/and sterile filtered.
38. The method and use according to any one of embodiments 20 to 37, wherein the solution comprising the alkyl polyglucoside detergent comprises a buffer salt.
39. The method and use according to any one of embodiments 20 to 38, wherein the solution comprising the alkyl polyglucoside detergent comprises TRIS.
40. The method and use according to any one of embodiments 20 to 39, wherein the solution comprising the alkyl polyglucoside detergent comprises a buffer salt has at a concentration of 100 mM to 1000 mM.
41. The method and use according to any one of embodiments 20 to 40, wherein the solution comprising the alkyl polyglucoside detergent comprises a buffer salt at a concentration of 250 mM to 750 mM.
42. The method and use according to any one of embodiments 20 to 41, wherein the solution comprising the alkyl polyglucoside detergent comprises a buffer salt at a concentration of 400 mM to 600 mM.
43. The method and use according to any one of embodiments 20 to 42, wherein the solution comprising the alkyl polyglucoside detergent comprises a buffer salt at a concentration of about 500 mM.
44. The method and use according to any one of embodiments 20 to 43, wherein the solution comprising the alkyl polyglucoside detergent comprises an ionic modifier salt.
45. The method and use according to any one of embodiments 20 to 44, wherein the solution comprising the alkyl polyglucoside detergent preferably comprises magnesium (II) chloride as ionic modifier salt.
46. The method and use according to any one of embodiments 20 to 45, wherein the solution comprising the alkyl polyglucoside detergent comprises an ionic modifier salt at a concentration of 5 mM to 200 mM.
47. The method and use according to any one of embodiments 20 to 46, wherein the solution comprising the alkyl polyglucoside detergent comprises an ionic modifier salt at a concentration of 7.5 mM to 150 mM.
48. The method and use according to any one of embodiments 20 to 47, wherein the solution comprising the alkyl polyglucoside detergent comprises an ionic modifier salt at a concentration of 10 mM to 50 mM.
49. The method and use according to any one of embodiments 20 to 48, wherein the solution comprising the alkyl polyglucoside detergent preferably comprises an ionic modifier salt at a concentration of 10 mM to 40 mM.
50. The method and use according to any one of the preceding embodiments, wherein bringing into contact or the contacting, respectively, with the alkyl polyglucoside detergent is at a pH value of from pH 6.5-9.0.
51. The method and use according to any one of the preceding embodiments, wherein bringing into contact or the contacting, respectively, with the alkyl polyglucoside detergent is at a pH value of from pH 6.5-8.0.
52. The method and use according to any one of the preceding embodiments, wherein the bringing into contact or the contacting, respectively, with the alkyl polyglucoside detergent is at a pH value of from pH 7.0-7.5.
53. The method and use according to any one of the preceding embodiments, wherein bringing into contact or the contacting, respectively, with the alkyl polyglucoside detergent is at a pH value of from pH 6.5-9.0 and the pH value is adjusted to a pH of 7.0-7.5 after the bringing into contact or the contacting, respectively.
54. The method and use according to any one of the preceding embodiments, wherein bringing into contact or the contacting, respectively, with the alkyl polyglucoside detergent is at a pH value of from pH 6.5-8.0 and the pH value is adjusted to a pH of 7.0-7.5 after the bringing into contact or the contacting, respectively.
55. The method and use according to any one of the preceding embodiments, wherein bringing into contact or the contacting, respectively, with the alkyl polyglucoside detergent is at a pH value of from pH 7.0-7.5 and the pH value is adjusted to a pH of about 7.5 after the bringing into contact or the contacting, respectively.
56. The method and use according to any one of embodiments 20 to 55, wherein the solution comprising the alkyl polyglucoside detergent has a pH value of pH 7.2 to 7.8.
57. The method and use according to any one of embodiments 20 to 56, wherein the solution comprising the alkyl polyglucoside detergent has a pH value of 7.3 to 7.7.
58. The method and use according to any one of embodiments 20 to 57, wherein the solution comprising the alkyl polyglucoside detergent has a pH value of 7.4 to 7.6.
59. The method and use according to any one of embodiments 20 to 58, wherein the solution comprising the alkyl polyglucoside detergent preferably has a pH value of about 7.5.
60. The method and use according to any one of the preceding embodiments, wherein the mammalian cell cultivation broth is brought in contact with the alkyl polyglucoside detergent and a nuclease.
61. The method and use according to any one of the preceding embodiments, wherein the mammalian cell cultivation broth is brought in contact with the alkyl polyglucoside detergent and DNase I.
62. The method and use according to any one of the preceding embodiments, wherein the mammalian cell cultivation broth is preferably brought in contact with the alkyl polyglucoside detergent and Benzonase.
63. The method and use according to any one of embodiments 60 to 62, wherein 25 to 100 U/mL nuclease is added.
64. The method and use according to any one of embodiments 60 to 63, wherein preferably 50 U/mL of each nuclease is added.
65. The method and use according to any one of the preceding embodiments, wherein the recombinant AAV particle-producing cell has been obtained by transfection with PEI.
66. The method and use according to any one of the preceding embodiments, wherein the affinity chromatography is on a chromatography material comprising a crosslinked poly(styrene-divinyl benzene) matrix to which an affinity ligand is covalently conjugated.
67. The method and use according to any one of the preceding embodiments, wherein the affinity chromatography is on a chromatography material comprising a crosslinked poly(styrene-divinyl benzene) matrix to which an affinity ligand is covalently conjugated.
68. The method and use according embodiment 67, wherein the affinity ligand is a single-domain antibody fragment (VHH).
69. The method and use according to any one of embodiments 67 to 68, wherein the affinity ligand specifically binds to the AAV serotypes AAV1, AAV2, AAV3, AAV4, AAV5, AAV6, AAV7, AAV8, AAVrh10 and synthetic serotypes based thereon.
70. The method and use according to any one of the preceding claims, wherein the affinity chromatography is on a chromatography material comprising a crosslinked poly(styrene-divinyl benzene) matrix to which a single-domain antibody fragment (VHH) specifically binding to the AAV serotypes AAV1, AAV2, AAV3, AAV4, AAV5, AAV6, AAV7, AAV8, AAVrh10 and synthetic serotypes based thereon is covalently conjugated.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### DETAILED DESCRIPTION OF THE INVENTION

The current invention is based at least in part on the finding that the recovery, i.e. yield, of recombinant AAV particles (both capsid-based yield as well as genome-based yield) in an AAV affinity chromatography is influenced/is depending on the detergent used for lysing the cells that produced the recombinant AAV particle prior to the AAV affinity chromatography.

The current invention is further based at least in part on the finding that the ratio of full recombinant AAV particles to empty recombinant AAV particles, which are obtained in an AAV affinity chromatography, is influenced/is depending on the detergent used for lysing the cells that produced the recombinant AAV particle prior to the AAV affinity chromatography.

It has been found that both (capsid-based as well as genomic) yield as well as the ratio of full to empty recombinant AAV particles can be increased in an AAV affinity chromatography by using an alkyl polyglucoside detergent for lysing the cells that produced the recombinant AAV particle prior to the AAV affinity chromatography step.

### Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “AAV helper functions” denotes AAV-derived coding sequences (proteins) which can be expressed to provide AAV gene products and AAV particles that, in turn, function in trans for productive AAV replication and packaging. Thus, AAV helper functions include AAV open reading frames (ORFs), including rep and cap and others such as AAP for certain AAV serotypes. The rep gene expression products have been shown to possess many functions, including, among others: recognition, binding and nicking of the AAV origin of DNA replication; DNA helicase activity; and modulation of transcription from AAV (or other heterologous) promoters. The cap gene expression products (capsids) supply necessary packaging functions. AAV helper functions are used to complement AAV functions in trans that are missing from AAV vector genomes.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The terms “comprise(s),” “include(s),” “having,” “has,” “can,” “contain(s)” and variants thereof, as used herein, are intended to be open-ended transitional phrases, terms or words that do not preclude the possibility of additional acts or structures. The term “comprising” also encompasses the term “consisting of”. The present disclosure also contemplates other embodiments “comprising,” “consisting of” and “consisting essentially of,” the embodiments or elements presented herein, whether explicitly set forth or not.

The term “cultivate” as used herein refers to the step of maintaining cells in a cultivation medium under conditions for the cells to be transfected and to produce AAV particles.

The term “culture broth” as used herein denotes the content of a bioreactor at the end of a cultivation. A “culture broth” comprises mammalian cells, dead and alive, mammalian cell debris, cultivation medium, recombinant product produced by the mammalian cells as well as other metabolic products produced by the mammalian cells.

The terms “empty particle” and “empty recombinant AAV particle”, which can be used interchangeably, denote an AAV particle that has an AAV protein shell but that lacks in whole or in part a nucleic acid that encodes a protein or is transcribed into a transcript of interest flanked by AAV ITRs, i.e. a vector. Accordingly, the empty particle does not function to transfer a nucleic acid that encodes a protein or is transcribed into a transcript of interest into a target cell.

The term “endogenous” denotes that something is naturally occurring within a cell; naturally produced by a cell; likewise, an “endogenous gene locus/cell-endogenous gene locus” is a naturally occurring locus in a cell.

As used herein, the term “exogenous” indicates that a nucleotide sequence does not originate from a specific cell and is introduced into said cell by DNA delivery methods, e.g., by transfection, electroporation, or transduction by viral vectors. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the sequence is becoming an “exogenous” sequence by its introduction into the cell, e.g., via recombinant DNA technology.

The term “fed-batch cell culture,” as used herein refers to a culture wherein the cells and culture medium are supplied to the culturing bioreactor initially, and additional culture nutrients are fed, continuously or in discrete increments, to the culture during the culturing process, with or without periodic cell and/or product harvest before termination of culture.

The terms “full particle” and “full recombinant AAV particle”, which can be used interchangeably, denote an AAV particle that has an AAV protein shell and therein encapsidated a nucleic acid that encodes a protein or is transcribed into a transcript of interest flanked by AAV ITRs, i.e. a vector. Accordingly, the full particle can transfer the encapsidated nucleic acid that encodes a protein or is transcribed into a transcript of interest into a target cell.

The terms “full to empty ratio” and “full recombinant AAV particle to empty recombinant AAV particle ratio”, which can be used interchangeably, denotes the mathematical ratio of the number of full recombinant AAV particles to the total number of recombinant AAV particles (full and empty) in a recombinant AAV particle containing sample or in a recombinant AAV particle preparation. As the number of full recombinant AAV particles can be at most the same as the total number of recombinant AAV particles the ratio can be at most 1. Generally, the ratio is less than 1 and is expressed as percentage. The number of full recombinant AAV particles is determined by determining the number of recombinant AAV particle encapsidated nucleic acid in the sample or preparation. This can be done by PCR, especially digital droplet PCR (ddPCR). The total number of recombinant AAV particles is determined by determining the number capsid proteins in the sample or preparation. This can be done by ELISA, especially by a capsid protein specific ELISA.

The “nucleic acids encoding AAV packaging proteins” refer generally to one or more nucleic acid molecule(s) that includes nucleotide sequences providing AAV functions deleted from an AAV vector, which is (are) to be used to produce a transduction competent recombinant AAV particle. The nucleic acids encoding AAV packaging proteins are commonly used to provide expression of AAV rep and/or cap genes to complement missing AAV functions that are necessary for AAV replication; however, the nucleic acid constructs lack AAV ITRs and can neither replicate nor package themselves. Nucleic acids encoding AAV packaging proteins can be in the form of a plasmid, phage, transposon, cosmid, virus, or particle. A number of nucleic acid constructs have been described, such as the commonly used plasmids pAAV/Ad and pIM29+45, which encode both rep and cap gene expression products. See, e.g., Samulski et al., J. Virol. 63 (1989) 3822-3828; and McCarty et al., J. Virol. 65 (1991) 2936-2945. A number of plasmids have been described which encode rep and/or cap gene expression products (e.g., U.S. Pat. Nos. 5,139,941 and 6,376,237). Any one of these nucleic acids encoding AAV packaging proteins can comprise the DNA element or nucleic acid according to the invention.

The term “nucleic acids encoding helper proteins” refers generally to one or more nucleic acid molecule(s) that include nucleotide sequences encoding proteins and/or RNA molecules that provide adenoviral helper function(s). A plasmid with nucleic acid(s) encoding helper protein(s) can be transfected into a suitable cell, wherein the plasmid is then capable of supporting AAV particle production in said cell. Any one of these nucleic acids encoding helper proteins can comprise the DNA element or nucleic acid according to the invention. Expressly excluded from the term are infectious viral particles, as they exist in nature, such as adenovirus, herpesvirus or vaccinia virus particles.

As used herein, the term “operably linked” refers to a juxtaposition of two or more components, wherein the components are in a relationship permitting them to function in their intended manner. For example, a promoter and/or an enhancer is operably linked to a coding sequence/open reading frame/gene if the promoter and/or enhancer acts to modulate the transcription of the coding sequence/open reading frame/gene. In certain embodiments, DNA sequences that are “operably linked” are contiguous. In certain embodiments, e.g., when it is necessary to join two protein encoding regions, such as a secretory leader and a polypeptide, the sequences are contiguous and in the same reading frame. In certain embodiments, an operably linked promoter is located upstream of the coding sequence/open reading frame/gene and can be adjacent to it. In certain embodiments, e.g., with respect to enhancer sequences modulating the expression of a coding sequence/open reading frame/gene, the two components can be operably linked although not adjacent. An enhancer is operably linked to a coding sequence/open reading frame/gene if the enhancer increases transcription of the coding sequence/open reading frame/gene. Operably linked enhancers can be located upstream, within, or downstream of coding sequences/open reading frames/genes and can be located at a considerable distance from the promoter of the coding sequence/open reading frame/gene.

The term “packaging proteins” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-I) and vaccinia virus.

As used herein, “AAV packaging proteins” refer to AAV-derived sequences, which function in trans for productive AAV replication. Thus, AAV packaging proteins are encoded by the major AAV open reading frames (ORFs), rep and cap. The rep proteins have been shown to possess many functions, including, among others: recognition, binding and nicking of the AAV origin of DNA replication; DNA helicase activity; and modulation of transcription from AAV (or other heterologous) promoters. The cap (capsid) proteins supply necessary packaging functions. AAV packaging proteins are used herein to complement AAV functions in trans that are missing from AAV vectors.

The term “recombinant cell” as used herein denotes a cell after final genetic modification, such as, e.g., a cell expressing a polypeptide of interest or producing a rAAV particle of interest and that can be used for the production of said polypeptide of interest or rAAV particle of interest at any scale. For example, “a mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”. Although the cell is still capable of performing further RMCE reactions, it is not intended to do so.

A “recombinant AAV vector” is derived from the wild-type genome of a virus, such as AAV by using molecular biological methods to remove the wild type genome from the virus (e.g., AAV), and replacing it with a non-native nucleic acid, such as a nucleic acid transcribed into a transcript or that encodes a protein. Typically, for AAV one or both inverted terminal repeat (ITR) sequences of the wild-type AAV genome are retained in the recombinant AAV vector. A “recombinant” AAV vector is distinguished from a wild-type viral AAV genome, since all or a part of the viral genome has been replaced with a non-native (i.e., heterologous) sequence with respect to the viral genomic nucleic acid. Incorporation of a non-native sequence therefore defines the viral vector (e.g., AAV) as a “recombinant” vector, which in the case of AAV can be referred to as a “rAAV vector.”

A recombinant vector (e.g., AAV) sequence can be packaged-referred to herein as a “particle”—for subsequent infection (transduction) of a cell, ex vivo, in vitro or in vivo. Where a recombinant vector sequence is encapsulated or packaged into an AAV particle, the particle can also be referred to as a “rAAV”. Such particles include proteins that encapsulate or package the vector genome. Particular examples include viral envelope proteins, and in the case of AAV, capsid proteins, such as AAV VP1, VP2 and VP3.

As used herein, the term “serotype” is a distinction based on AAV capsids being serologically distinct. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

The term “transgene” is used herein to conveniently refer to a nucleic acid that is intended or has been introduced into a cell or organism. Transgenes include any nucleic acid, such as a gene that is transcribed into a transcript or that encodes a polypeptide or protein.

The term “Triton CG 110” denotes an alkyl polyglucoside detergent. Triton CG 110 has the CAS number 68515-73-1. Triton CG 110 is a mixture of 58.0-62.0 (w/v) % D-glucopyranose, oligomeric, decyl octyl glycoside and 38.0-42.0 (w/v) % water.

A “vector” refers to the portion of the recombinant plasmid sequence ultimately packaged or encapsulated, either directly or in form of a single strand or RNA, to form a viral (e.g., AAV) particle. In cases recombinant plasmids are used to construct or manufacture recombinant viral particles, the viral particle does not include the portion of the “plasmid” that does not correspond to the vector sequence of the recombinant plasmid. This non-vector portion of the recombinant plasmid is referred to as the “plasmid backbone”, which is important for cloning and amplification of the plasmid, a process that is needed for propagation and recombinant virus production, but is not itself packaged or encapsulated into virus (e.g., AAV) particles. Thus, a “vector” refers to the nucleic acid that is packaged or encapsulated by a virus particle (e.g., AAV).

### Recombinant Cell

Generally, for efficient as well as large-scale production of a recombinant AAV particle (rAAV particle) a cell expressing and, if possible, also secreting said rAAV particle. Such a cell is termed “recombinant cell” or “recombinant production cell”.

For the generation of a “recombinant production cell” a suitable mammalian cell is transfected with the required nucleic acid sequences for producing said rAAV particle, including the required AAV helper functions.

For expression of a coding sequence, i.e. of an open reading frame, additional regulatory elements, such as a promoter and polyadenylation signal (sequence), are necessary. Thus, an open reading frame is operably linked to said additional regulatory elements for transcription. This can be achieved by integrating it into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case of an AAV particle, which is composed of different (monomeric) capsid polypeptides and a single stranded DNA molecule and which in addition requires other adenoviral helper functions for production and encapsulation, a multitude of expression cassettes differing in the contained open reading frames/coding sequences are required. In this case, at least an expression cassette for each of the transgene, the different polypeptides forming the capsid of the AAV vector, for the required helper functions as well as the VA RNA are required. Thus, individual expression cassettes for each of the helper functions E1A, E1B, E2A, E4orf6, the VA RNA, the rep and cap genes are required. HEK293 cells express the E1A and E1B helper functions constitutively.

In certain embodiments of all aspects and embodiments of the invention, each of the expression cassettes comprise in 5′-to-3′ direction a promoter, an open reading frame/coding sequence or an RNA gene and a polyadenylation signal sequence, and/or a terminator sequence. In certain embodiments, the open reading frame encodes a polypeptide and the expression cassette comprises a polyadenylation signal sequence with or without additional terminator sequence. In certain embodiments, the expression cassette comprises a RNA gene, the promoter is a type 2 Pol III promoter and a polyadenylation signal sequence or a polyU terminator is present. See, e.g., Song et al. Biochemical and Biophysical Research Communications 323 (2004) 573-578. In certain embodiments, the expression cassette comprises a RNA gene, the promoter is a type 2 Pol III promoter and a polyU terminator sequence.

In certain embodiments of all aspects and embodiments of the invention, the open reading frame encodes a polypeptide, the promoter is the human CMV promoter with or without intron A, the polyadenylation signal sequence is the bGH (bovine growth hormone) polyA signal sequence and the terminator is the hGT (human gastrin terminator).

In certain embodiments of all aspects and embodiments of the invention, the promoter is the human CMV promoter with intron A, the polyadenylation signal sequence is the bGH polyadenylation signal sequence and the terminator is the hGT, except for the expression cassette of the RNA gene and the expression cassette of the selection marker, wherein for the selection marker the promoter is the SV40 promoter and the polyadenylation signal sequence is the SV40 polyadenylation signal sequence and a terminator is absent, and wherein for the RNA gene the promoter is a wild-type type 2 polymerase III promoter and the terminator is a polymerase II or III terminator.

### Adeno-Associated Virus (AAV)

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV vectors are described (see also WO 93/24641). Additional references describing AAV vectors are West et al., Virol. 160 (1987) 38-47; Kotin, Hum. Gene Ther. 5 (1994) 793-801; and Muzyczka J. Clin. Invest. 94 (1994) 1351. Construction of recombinant AAV vectors described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828.

An adeno-associated virus (AAV) is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin provided that the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV-2 other integration sites have been found, such as, e.g., on chromosome 5 [(Chr) 5 (p13.3)], termed AAVS2, and on chromosome 3 [(Chr) 3 (p24.3)], termed AAVS3.

AAVs are categorized into different serotypes. These have been allocated based on parameters, such as hemagglutination, tumorigenicity and DNA sequence homology. Up to now, more than 10 different serotypes and more than a hundred sequences corresponding to different clades of AAV have been identified.

The capsid protein type and symmetry determines the tissue tropism of the respective AAV. For example, AAV-2, AAV-4 and AAV-5 are specific to retina, AAV-2, AAV-5, AAV-8, AAV-9 and AAVrh-10 are specific for brain, AAV-1, AAV-2, AAV-6, AAV-8 and AAV-9 are specific for cardiac tissue, AAV-1, AAV-2, AAV-5, AAV-6, AAV-7, AAV-8, AAV-9 and AAV-10 are specific for liver, AAV-1, AAV-2, AAV-5 and AAV-9 are specific for lung.

Pseudotyping denotes a process comprising the cross packaging of the AAV genome between various serotypes, i.e. the genome is packaged with differently originating capsid proteins.

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding open reading frame provides for four proteins of different size, which are termed Rep78, Rep68, Rep52 and Rep40. These are involved in replication, rescue and integration of the AAV. The Cap protein encoding open reading frame provides four proteins, which are termed VP1, VP2, VP3, and AAP. VP1, VP2 and VP3 are part of the proteinaceous capsid of the AAV particles. The combined rep and cap open reading frames are flanked at their 5′- and 3′-ends by so-called inverted terminal repeats (ITRs). For replication, an AAV requires in addition to the Rep and Cap proteins the products of the genes E1A, E1B, E4orf6, E2A and VA of an adenovirus or corresponding factors of another helper virus.

In the case of an AAV of the serotype 2 (AAV-2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The AAV genome harbors three transcription promoters P5, P19, and P40 (Laughlin et al., Proc. Natl. Acad. Sci. USA 76 (1979) 5567-5571) for the expression of the rep and cap genes.

The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter in an AAV vector (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome an in trans organization of the rep and cap gene products are required.

The rep gene locus comprises two internal promoters, termed P5 and P19. It comprises open reading frames for four proteins. Promoter P5 is operably linked to a nucleic acid sequence providing for non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The two larger Rep proteins, Rep78 and Rep68, are essential for AAV duplex DNA replication, whereas the smaller Rep proteins, Rep52 and Rep40, seem to be essential for progeny, single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene locus comprises one promoter, termed P40. Promoter P40 is operably linked to a nucleic acid sequence providing for 2.6 kb mRNA, which, by alternative splicing and use of alternative start codons, encodes the Cap proteins VP1 (87 kDa, non-spliced mRNA transcript), VP2 (72 kDa, from the spliced mRNA transcript), and VP3 (61 kDa, from alternative start codon). VP1 to VP3 constitute the building blocks of the viral capsid. The capsid has the function to bind to a cell surface receptor and allow for intracellular trafficking of the virus. VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is contained. This may be either the “plus” or “minus” strand. AAV viral particles containing a DNA molecule are infectious. Inside the infected cell, the parental infecting single strand is converted into a double strand, which is subsequently amplified. The amplification results in a large pool of double stranded DNA molecules from which single strands are displaced and packaged into capsids.

Adeno-associated viral (AAV) vectors can transduce dividing cells as well as resting cells. It can be assumed that a transgene introduced using an AAV vector into a target cell will be expressed for a long period. One drawback of using an AAV vector is the limitation of the size of the transgene that can be introduced into cells.

Viral vectors such as parvo-virus particles, including AAV serotypes and variants thereof, provide a means for delivery of nucleic acid into cells ex vivo, in vitro and in vivo, which encode proteins such that the cells express the encoded protein. AAVs are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example. Because AAV are not associated with pathogenic disease in humans, AAV vectors are able to deliver heterologous polynucleotide sequences (e.g., therapeutic proteins and agents) to human patients without causing substantial AAV pathogenesis or disease.

AAV particles used as vehicles for effective gene delivery possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

Recombinant AAV particles do not typically include viral genes associated with pathogenesis. Such vectors typically have one or more of the wild-type AAV genes deleted in whole or in part, for example, rep and/or cap genes, but retain at least one functional flanking ITR sequence, as necessary for the rescue, replication, and packaging of the recombinant vector into an AAV particle. For example, only the essential parts of the vector e.g., the ITR and LTR elements, respectively, are included. An AAV vector genome would therefore include sequences required in cis for replication and packaging (e.g., functional ITR sequences).

Recombinant AAV vectors, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, 218, AAV rh74 or AAV 7m8 for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be an AAV1, AAV3, AAV4, AAV5, AAV6, AAV7, AAV8, AAV9, AAV10, AAV11, AAV12, AAV-218, AAV rh74, AAV 7m8 or a variant thereof, for example. AAV variants include variants and chimeras of AAV1, AAV2, AAV3, AAV4, AAV5, AAV6, AAV7, AAV8, AAV9, AAV10, AAV11, AAV12, AAV-218, AAV rh74 and AAV 7m8 capsids.

In certain embodiments of all aspects and embodiments of the invention, the rAAV particle is derived from an AAV selected from the group consisting of AAV1, AAV2, AAV3, AAV4, AAV5, AAV6, AAV7, AAV8, AAV9, AAV10, AAV11, AAV12, AAV-218, AAV rh74 and AAV 7m8, as well as variants (e.g., capsid variants, such as amino acid insertions, additions, substitutions and deletions) thereof, for example, as set forth in WO 2013/158879, WO 2015/013313 and US 2013/0059732 (disclosing LK01, LK02, LK03, etc.).

In certain embodiments of all aspects and embodiments of the invention, the rAAV particle comprises a capsid sequence having 70% or more sequence identity to an AAV1, AAV2, AAV3, AAV4, AAV5, AAV6, AAV7, AAV8, AAV9, AAV10, AAVrh10, AAV Rh74, or AAV 7m8 capsid sequence.

In certain embodiments of all aspects and embodiments of the invention, the rAAV particle comprises an ITR sequence having 70% or more sequence identity to an AAV1, AAV2, AAV3, AAV4, AAV5, AAV6, AAV7, AAV8, AAV9, or AAV10 ITR sequence

Recombinant particles (e.g., rAAV particles) can be incorporated into pharmaceutical compositions. Such pharmaceutical compositions are useful for, among other things, administration and delivery to a subject in vivo or ex vivo. In certain embodiments, the pharmaceutical composition contains a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744, which are incorporated herein by reference in their entirety.

Recombinant AAV Particles (rAAV Particles)

Different methods that are known in the art for generating rAAV particles. For example, transfection using AAV plasmid and AAV helper sequences in conjunction with co-infection with one AAV helper virus (e.g., adenovirus, herpesvirus, or vaccinia virus) or transfection with a recombinant AAV plasmid, an AAV helper plasmid, and an helper function plasmid. Non-limiting methods for generating rAAV particles are described, for example, in U.S. Pat. Nos. 6,001,650, 6,004,797, WO 2017/096039, and WO 2018/226887. Following recombinant rAAV particle production (i.e. particle generation in cell culture systems), rAAV particles can be obtained from the host cells and cell culture supernatant and purified.

For the generation of recombinant AAV particles, expression of the Rep and Cap proteins, the helper proteins E1A, E1B, E2A and E4orf6 as well as the adenoviral VA RNA in a single mammalian cell is required. The helper proteins E1A, E1B, E2A and E4orf6 can be expressed using any promoter as shown by Matsushita et al. (Gene Ther. 5 (1998) 938-945), especially the CMV IE promoter. Thus, any promoter can be used.

Generally, to produce recombinant AAV particles, different, complementing plasmids are co-transfected into a host cell. One of the plasmids comprises the transgene sandwiched between the two cis acting AAV ITRs. The missing AAV elements required for replication and subsequent packaging of progeny recombinant genomes, i.e. the open reading frames for the Rep and Cap proteins, are contained in trans on a second plasmid. The overexpression of the Rep proteins results in inhibitory effects on cell growth (Li, J., et al., J. Virol. 71 (1997) 5236-5243). Additionally, a third plasmid comprising the genes of a helper virus, i.e. E1, E4orf6, E2A and VA from adenovirus, is required for AAV replication.

To reduce the number of required plasmids, Rep, Cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrid vectors can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

In order to limit the transgene activity to specific tissues, i.e. to limit the site of integration the transgene can be operably linked to an inducible or tissue specific promoter (see, e.g., Yang, Y., et al. Hum. Gene. Ther. 6 (1995) 1203-1213).

### E1A, E1B, E2 and E4

The coding sequences of E1A and E1B (open reading frames) can be derived from a human adenovirus, such as, e.g., in particular of human adenovirus serotype 2 or serotype 5. An exemplary sequence of human Ad5 (adenovirus serotype 5) is found in GenBank entries X02996, AC_000008 and that of an exemplary human Ad2 in GenBank entry AC_000007. Nucleotides 505 to 3522 comprise the nucleic acid sequences encoding E1A and E1B of human adenovirus serotype 5. Plasmid pSTK146 as reported in EP 1 230 354, as well as plasmids pGS119 and pGS122 as reported in WO 2007/056994, can also be used a source for the E1A and E1B open reading frames.

E1A is the first viral helper gene that is expressed after adenoviral DNA enters the cell nucleus. The ELA gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will die (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis. The E1B 19 kDa protein is involved in the prevention of E1A-induced apoptosis of cells.

The E2 gene encodes different proteins. The E2A transcript codes for the single strand-binding protein (SSBP), which is essential for AAV replication

Likewise, the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

### Adenoviral VA RNA Gene

The viral associated RNA (VA RNA) is a non-coding RNA of adenovirus (Ad), regulating translation. The adenoviral genome comprises two independent copies: VAI (VA RNAI) and VAII (VA RNAII). Both are transcribed by RNA polymerase III (see, e.g., Machitani, M., et al., J. Contr. Rel. 154 (2011) 285-289) from a type 2 polymerases III promoter. For recombinant production, the adenoviral VA RNA gene can be driven by any promoter.

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes. Said disclosure is herewith incorporated by reference in its entirety into the current application.

VA RNAs, VAI and VAII, are consisting of 157-160 nucleotides (nt).

Depending on the serotype, adenoviruses contain one or two VA RNA genes. VA RNAI is believed to play the dominant pro-viral role, while VA RNAII can partially compensate for the absence of VA RNAI (Vachon, V. K. and Conn, G. L., Virus Res. 212 (2016) 39-52).

The VA RNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

Maekawa, A., et al. (Nature Sci. Rep. 3 (2013) 1136) reported efficient production of adenovirus vector lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI corresponds to nucleotides 10586-10810 of GenBank entry AC_000007 sequence. The human adenovirus 5 VA RNAI corresponds to nucleotides 10579-10820 of GenBank entry AC_000008 sequence.

Methods for Producing rAAV Particles

Carter et al. have shown that the entire rep and cap open reading frames in the wild-type AAV genome can be deleted and replaced with a transgene (Carter, B. J., in “Handbook of Parvoviruses”, ed. by P. Tijssen, CRC Press, pp. 155-168 (1990)). Further, it has been reported that the ITRs have to be maintained to retain the function of replication, rescue, packaging, and integration of the transgene into the genome of the target cell.

When cells comprising the respective viral helper genes are transduced by an AAV vector, or, vice versa, when cells comprising an integrated AAV provirus are transduced by a suitable helper virus, then the AAV provirus is activated and enters a lytic infection cycle again (Clark, K. R., et al., Hum. Gene Ther. 6 (1995) 1329-1341; Samulski, R. J., Curr. Opin. Genet. Dev. 3 (1993) 74-80).

Producer cells contain the rep and cap gene sequences, as well as the transgene cassette flanked by ITR sequences on one or more plasmids that are retained via drug selection. Production of rAAV particles in these cell lines generally occurs after their infection with the required helper functions. Therefore, cells are infected either with replication-competent AdV (usually wild type Ad5) or a plasmid comprising the respective helper genes to supply helper virus proteins and initiate rAAV particle production. A packaging cell line differs from a producer cell line as it only contains the rep and cap genes.

The methods according to the current invention include the step of transducing a mammalian cell with nucleic acids (e.g., plasmids) comprising all required elements for the production of recombinant AAV particles. Thus, as the plasmids encode viral packaging proteins and/or helper proteins the cells can produce recombinant viral particles that include a nucleic acid that encodes a protein of interest or comprises a sequence that is transcribed into a transcript of interest.

The invention provides a recombinant AAV viral particle production platform that includes features that distinguish it from current ‘industry-standard’ recombinant AAV particle production processes including the lysis step according to the current invention.

More generally, cells transfected or transduced with DNA for the recombinant production of AAV particles can be referred to as a “recombinant cell”. Such a cell can be any mammalian cell that has been used as recipient of a nucleic acid (plasmid) encoding packaging proteins, such as AAV packaging proteins, a nucleic acid (plasmid) encoding helper proteins, and a nucleic acid (plasmid) that encodes a protein or is transcribed into a transcript of interest, i.e. a transgene placed between two AAV ITRs. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth media appropriate for sustaining cell viability or providing cell growth and/or proliferation are commercially available. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such media can be supplemented with vitamins and/or trace minerals and/or salts and/or amino acids, such as essential amino acids for mammalian (e.g., human) cells.

Thus, herein is provided a method for producing recombinant AAV vectors or AAV particles comprising said recombinant AAV vectors, which comprise a nucleic acid that encodes a protein or is transcribed into a transcript of interest, using the lysis step according to the current invention.

For this purpose, three plasmids are co-transfected into a mammalian cell. The transgene plasmid encodes the expression cassette, which is cloned between the AAV ITRs, whereas rep and cap genes are provided in trans by co-transfecting a second, packaging plasmid (rep/cap plasmid) to ensure AAV replication and packaging. The third plasmid, also referred to as helper plasmid, contains the minimal helper virus factors, commonly adenoviral E2A, EV and VA genes, but lacking the AAV ITRs.

One aspect of the current invention is a method for producing recombinant AAV vectors or AAV particles comprising said recombinant AAV vectors, which comprise a nucleic acid that encodes a protein or is transcribed into a transcript of interest, comprising the steps of
(i) providing one or more plasmids comprising nucleic acids encoding AAV packaging proteins and/or nucleic acids encoding helper proteins;
(ii) providing a plasmid comprising a nucleic acid that encodes a protein of interest or is transcribed into a transcript of interest that is interspaced between AAV ITRs;
(iii) contacting one or more mammalian cells with the provided plasmids and either further adding a transfection reagent and optionally incubating the plasmid/transfection reagent/cell mixture; or providing a physical means, such as an electric current, to introduce the nucleic acid into the cells;
(iv) cultivating the transfected cells;
(v) harvesting the cultivated cells and culture medium to produce a mammalian cell cultivation broth;
(vi) lysing the cells by bringing the mammalian cell cultivation broth in contact with an alkyl polyglucoside detergent to produce a mammalian cell cultivation broth lysate; and
(vii) optionally isolating recombinant AAV particles from the cultivation broth lysate with an AAV affinity chromatography;
thereby producing a recombinant AAV particle comprising a nucleic acid that encodes a protein of interest or is transcribed into a transcript of interest.

One aspect of the current invention is a method for producing recombinant AAV vectors or AAV particles comprising said recombinant AAV vectors, which comprise a nucleic acid that encodes a protein or is transcribed into a transcript of interest, comprising the steps of
(i) providing one or more plasmids comprising nucleic acids encoding AAV packaging proteins and/or nucleic acids encoding helper proteins;
(ii) providing a plasmid comprising a nucleic acid that encodes a protein of interest or is transcribed into a transcript of interest;
(iii) either
   (a) generating a stable transfected cell by contacting one or more mammalian cells with the provided plasmids of (i) by either further adding a transfection reagent and optionally incubating the plasmid/transfection reagent/cell mixture or providing a physical means, such as an electric current, to introduce the nucleic acid into the cells; selecting a first stably transfected cell; contacting the selected first stably transfected cell with the provided plasmid of (ii) and either further adding a transfection reagent and optionally incubating the plasmid/transfection reagent/cell mixture or providing a physical means, such as an electric current, to introduce the nucleic acid into the cells; or
   (b) generating a transient transfected cell by contacting one or more mammalian cells with the provided plasmids of (i) and (ii) and either further adding a 1 transfection reagent and optionally incubating the plasmid/transfection reagent/cell mixture, or providing a physical means, such as an electric current, to introduce the nucleic acid into the cells;
   thereby generating a transfected cell;
(iv) cultivating the transfected cell of (iii);
(v) harvesting the cultivated cells and the culture medium to produce a mammalian cell cultivation broth;
(vi) lysing the cells by bringing the mammalian cell cultivation broth in contact with an alkyl polyglucoside detergent to produce a mammalian cell cultivation broth lysate; and
(vii) optionally isolating recombinant AAV particles from the mammalian cell cultivation broth lysate with an AAV affinity chromatography;
thereby producing recombinant AAV particles comprising a nucleic acid that encodes a protein of interest or is transcribed into a transcript of interest.

The introduction of the nucleic acid (plasmids) into cells can be done in multiple ways.

Diverse methods for the DNA transfer into mammalian cells have been reported in the art. These are all useful in the methods according to the current invention. In certain embodiments of all aspects and embodiments, electroporation, nucleofection, or microinjection for nucleic acid transfer/transfection is used. In certain embodiments of all aspects and embodiments, an inorganic substance (such as, e.g., calcium phosphate/DNA co-precipitation), a cationic polymer (such as, e.g., polyethylenimine, DEAE-dextran), or a cationic lipid (lipofection) is used for nucleic acid transfer/transfection is used. Calcium phosphate and polyethylenimine are the most commonly used reagents for transfection for nucleic acid transfer in larger scales (see, e.g., Baldi et al., Biotechnol. Lett. 29 (2007) 677-684), whereof polyethylenimine is preferred.

The growth in serum-free suspension culture and improvement of efficiency and reproducibility of transfection conditions using PEI as a transfection reagent permits ready scale-up the AAV production using shake-flasks, wave, or stirred-tank bioreactors.

In certain embodiments of all aspects and embodiments, the nucleic acid (plasmid) is provided in a composition in combination with polyethylenimine (PEI), optionally in combination with cells. In certain embodiments, the composition includes a plasmid/PEI mixture, which has a plurality of components: (a) one or more plasmids comprising nucleic acids encoding AAV packaging proteins and/or nucleic acids encoding helper proteins; (b) a plasmid comprising a nucleic acid that encodes a protein or is transcribed into a transcript of interest; and (c) a polyethylenimine (PEI) solution. In certain embodiments, the plasmids are in a molar ratio range of about 1:0.01 to about 1:100, or are in a molar ratio range of about 100:1 to about 1:0.01, and the mixture of components (a), (b) and (c) has optionally been incubated for a period of time from about 10 seconds to about 4 hours.

In certain embodiments of all aspects and embodiments, the compositions further comprise cells. In certain embodiments, the cells are in contact with the plasmid/PEI mixture of components (a), (b) and/or (c).

In certain embodiments of all aspects and embodiments, the composition, optionally in combination with cells, further comprise free PEI. In certain embodiments, the cells are in contact with the free PEI.

In certain embodiments of all aspects and embodiments, the cells have been in contact with the mixture of components (a), (b) and/or (c) for at least about 4 hours, or about 4 hours to about 140 hours, or for about 4 hours to about 96 hours. In one preferred embodiment, the cells have been in contact with the mixture of components (a), (b) and/or (c) and optionally free PEI, for at least about 4 hours.

The composition may comprise further plasmids or/and cells. Such plasmids and cells may be in contact with free PEI. In certain embodiments, the plasmids and/or cells have been in contact with the free PEI for at least about 4 hours, or about 4 hours to about 140 hours, or for about 4 hours to about 96 hours.

The method according to the invention also includes steps of transfecting cells. The methods, thus, include steps of providing one or more plasmids; providing a solution comprising polyethylenimine (PEI); and mixing the plasmid(s) with the PEI solution to produce a plasmid/PEI mixture. In certain embodiments, such mixtures are incubated for a period in the range of about 10 seconds to about 4 hours. In such methods, cells are then contacted with the plasmid/PEI mixture to produce a plasmid/PEI cell culture; then free PEI is added to the plasmid/PEI cell culture produced to produce a free PEI/plasmid/PEI cell culture; and then the free PEI/plasmid/PEI cell culture produced is incubated for at least about 4 hours, thereby producing transfected cells. In certain embodiments, the plasmids comprise one or more or all of a rep open reading frame, a cap open reading frame, E1A, E1B, E2 and E4orf6 open reading frames and a nucleic acid that encodes a protein or is transcribed into a transcript of interest.

Further the methods according to the invention includes steps for producing transfected cells that produce recombinant AAV vector or AAV particle, which include providing one or more plasmids comprising nucleic acids encoding AAV packaging proteins and/or nucleic acids encoding helper proteins; providing a plasmid comprising a nucleic acid that encodes a protein or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period in the range of about 10 seconds to about 4 hours); contacting mammalian cells with the plasmid/PEI mixture, to produce a plasmid/PEI cell culture; adding free PEI to the plasmid/PEI cell culture produced to produce a free PEI/plasmid/PEI cell culture; and incubating the free PEI/plasmid/PEI cell culture for at least about 4 hours, thereby producing transfected cells that produce recombinant AAV vector or particle comprising a nucleic acid that encodes a protein or is transcribed into a transcript of interest, whereby the mammalian cells have been obtained with a method according to the current invention.

Additionally provided are methods for producing a recombinant AAV vector or AAV particle comprising a nucleic acid that encodes a protein or is transcribed into a transcript of interest, which includes providing one or more plasmids comprising nucleic acids encoding AAV packaging proteins and/or nucleic acids encoding helper proteins; providing a plasmid comprising a nucleic acid that encodes a protein of interest or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period of time in the range of about 10 seconds to about 4 hours); contacting mammalian cells with the plasmid/PEI mixture produced as described to produce a plasmid/PEI cell culture; adding free PEI to the plasmid/PEI cell culture produced as described to produce a free PEI/plasmid/PEI cell culture; incubating the plasmid/PEI cell culture or the free PEI/plasmid/PEI cell culture produced for at least about 4 hours to produce transfected cells; harvesting the transfected cells produced and/or culture medium from the transfected cells produced to produce a cultivation broth; lysing the cells with a method according to the current invention and optionally isolating the recombinant AAV vector or particle from the cultivation broth lysate with an AAV affinity chromatography step; and thereby producing recombinant AAV vector or particle comprising a nucleic acid that encodes a protein or is transcribed into a transcript of interest.

In certain embodiments of all aspects and embodiments, PEI is added to the plasmids and/or cells at various time points. In certain embodiments, free PEI is added to the cells before, at the same time as, or after the plasmid/PEI mixture is contacted with the cells.

In certain embodiments of all aspects and embodiments, the cells are at particular densities and/or cell growth phases and/or viability when contacted with the plasmid/PEI mixture and/or when contacted with the free PEI. In one preferred embodiment, cells are at a density in the range of about 1*10\hat{ }5 cells/mL to about 1*10\hat{ }8 cells/mL when contacted with the plasmid/PEI mixture and/or when contacted with the free PEI. In certain embodiments, viability of the cells when contacted with the plasmid/PEI mixture or with the free PEI is about 60% or greater than 60%, or wherein the cells are in log phase growth when contacted with the plasmid/PEI mixture, or viability of the cells when contacted with the plasmid/PEI mixture or with the free PEI is about 90% or greater than 90%, or wherein the cells are in log phase growth when contacted with the plasmid/PEI mixture or with the free PEI.

In addition to PEI, valproic acid (VPA) can be used to improve transfection efficiency. VPA, a branched short-chain fatty acid and inhibits histone deacetylase activity. Due to this reason, it is commonly added to mammalian cell culture as an enhancer of recombinant protein production.

Encoded AAV packaging proteins include, in certain embodiments of all aspects and embodiments, AAV rep and/or AAV cap. Such AAV packaging proteins include, in certain embodiments of all aspects and embodiments, AAV rep and/or AAV cap proteins of any AAV serotype.

Encoded helper proteins include, in certain embodiments of all aspects and embodiments, adenovirus E1A and E1B, adenovirus E2 and/or E4, VA RNA, and/or non-AAV helper proteins.

In certain embodiments of all aspects and embodiments, the nucleic acids (plasmids) are used at particular amounts or ratios. In certain embodiments, the total amount of plasmid comprising the nucleic acid that encodes a protein or is transcribed into a transcript of interest and the one or more plasmids comprising nucleic acids encoding AAV packaging proteins and/or nucleic acids encoding helper proteins is in the range of about 0.1 μg to about 15 μg per mL of cells. In certain embodiments, the molar ratio of the plasmid comprising the nucleic acid that encodes a protein or is transcribed into a transcript of interest to the one or more plasmids comprising nucleic acids encoding AAV packaging proteins and/or nucleic acids encoding helper proteins is in the range of about 1:5 to about 1:1, or is in the range of about 1:1 to about 5:1.

In certain embodiments of all aspects and embodiments, a first plasmid comprises the nucleic acids encoding AAV packaging proteins and a second plasmid comprises the nucleic acids encoding helper proteins.

In certain embodiments of all aspects and embodiments, the molar ratio of the plasmid comprising the nucleic acid that encodes a protein or is transcribed into a transcript of interest to a first plasmid comprising the nucleic acids encoding AAV packaging proteins to a second plasmid comprising the nucleic acids encoding helper proteins is in the range of about 1-5:1:1, or 1:1-5:1, or 1:1:1-5 in co-transfection.

In certain embodiments of all aspects and embodiments, the cell is a mammalian cell. In one preferred embodiment, the cell is a HEK293 cell or a CHO cell.

The cultivation can be performed using the generally used conditions for the cultivation of eukaryotic cells of about 37° C., 95% humidity and 8 vol.-% CO₂. The cultivation can be performed in serum containing or serum free medium, in adherent culture or in suspension culture. The suspension cultivation can be performed in any fermentation vessel, such as, e.g., in stirred tank reactors, wave reactors, rocking bioreactors, shaker vessels or spinner vessels or so called roller bottles. Transfection can be performed in high throughput format and screening, respectively, e.g. in a 96 or 384 well format.

Methods according to the current invention can include AAV particles of any serotype, or a variant thereof. In certain embodiments of all aspects and embodiments, a recombinant AAV particle comprises any of AAV serotypes 1-12, an AAV VP1, VP2 and/or VP3 capsid protein, or a modified or variant AAV VP1, VP2 and/or VP3 capsid protein, or wild-type AAV VP1, VP2 and/or VP3 capsid protein. In certain embodiments of all aspects and embodiments, an AAV particle comprises an AAV serotype or an AAV pseudotype, where the AAV pseudotype comprises an AAV capsid serotype different from an ITR serotype.

Methods according to the invention that provide or include AAV vectors or particles can also include other elements. Examples of such elements include, but are not limited to, an intron, an expression control element, one or more adeno-associated virus (AAV) inverted terminal repeats (ITRs) and/or a filler/stuffer polynucleotide sequence. Such elements can be within or flank the nucleic acid that encodes a protein or is transcribed into a transcript of interest, or the expression control element can be operably linked to nucleic acid that encodes a protein or is transcribed into a transcript of interest, or the AAV ITR(s) can flank the 5′- or 3′-terminus of nucleic acid that encodes a protein or is transcribed into a transcript of interest, or the filler polynucleotide sequence can flank the 5′- or 3′-terminus of nucleic acid that encodes a protein or is transcribed into a transcript of interest.

Expression control elements include constitutive or regulatable control elements, such as a tissue-specific expression control element or promoter.

ITRs can be any of AAV2 or AAV6 or AAV8 or AAV9 serotypes, or a combination thereof. AAV particles can include any VP1, VP2 and/or VP3 capsid protein having 75% or more sequence identity to any of AAV1, AAV2, AAV3, AAV4, AAV5, AAV6, AAV10, AAV11, AAV-218, AAV rh74 or AAV 7m8 VP1, VP2 and/or VP3 capsid proteins, or comprises a modified or variant VP1, VP2 and/or VP3 capsid protein selected from any of: AAV1, AAV2, AAV3, AAV4, AAV5, AAV6, AAV10, AAV11, AAV-218, AAV rh74 and AAV 7m8 AAV serotypes.

Following production of recombinant viral (e.g., AAV) particles as set forth herein, if desired, the viral (e.g., rAAV) particles can be purified and/or isolated from host cells using a variety of conventional methods. Such methods include column chromatography, CsCl gradients, iodixanol gradient and the like.

For example, a plurality of column purification steps such as purification over an anion exchange column, an affinity column and/or a cation exchange column can be used. (See, e.g., WO 02/12455 and US 2003/0207439). Alternatively, or in addition, an iodixanol or CsCl gradient steps can be used (see, e.g., US 2012/0135515; and US 2013/0072548). Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

An objective in the rAAV vector production and purification systems is to implement strategies to minimize/control the generation of production related impurities such as proteins, nucleic acids, and vector-related impurities, including wild-type/pseudo wild-type AAV species (wtAAV) and AAV-encapsulated residual DNA impurities.

Considering that the rAAV particle represents only a minor fraction of the biomass, rAAV particles need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

As an initial step, typically the cultivated cells that produce the rAAV particles are harvested, optionally in combination with harvesting cell culture supernatant (medium) in which the cells (suspension or adherent) producing rAAV particles have been cultured. The harvested cells and optionally cell culture supernatant may be used as is, as appropriate, lysed or concentrated. Further, if infection is employed to express helper functions, residual helper virus can be inactivated. For example, adenovirus can be inactivated by heating to temperatures of approximately 60° C. for, e.g., 20 minutes or more, which inactivates only the helper virus since AAV is heat stable while the helper adenovirus is heat labile.

The cells in the harvested cultivation broth are lysed by the method according to the current invention to release the rAAV particles. Concurrently during cell lysis or subsequently after cell lysis, a nuclease, such as, e.g., benzonase, is added to degrade contaminating DNA. Typically, the resulting lysate is clarified to remove cell debris, e.g. by filtering or centrifuging, to render a clarified cell lysate. In a particular example, lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains AAV particles (comprising rAAV vectors as well as empty capsids) and production/process related impurities, such as soluble cellular components from the host cells that can include, inter alia, cellular proteins, lipids, and/or nucleic acids, and cell culture medium components. The optionally clarified lysate is then subjected to purification steps to purify AAV particles (comprising rAAV vectors) from impurities using chromatography. The clarified lysate may be diluted or concentrated with an appropriate buffer prior to the first chromatography step.

After cell lysis, optional clarifying, and optional dilution or concentration, a plurality of subsequent and sequential chromatography steps can be used to purify rAAV particles.

The first chromatography step is preferably an affinity chromatography step using an AAV affinity chromatography ligand.

If the first chromatography step is affinity chromatography the second chromatography step can be anion exchange chromatography. Thus, in certain embodiments of all aspects and embodiments, rAAV particle purification is via affinity chromatography, followed by purification via anion exchange chromatography or/and cation exchange chromatography or/and size exclusion chromatography, in any order or sequence or combination.

The removal of empty capsids from full ones, for example, during downstream processing is based on their different isoelectric points (pI) in anion exchange chromatography. The average calculated pI across all serotypes is 5.9 for full capsids and 6.3 for empty capsids (Venkatakrishnan, B., et al., J. Virol. 87 (2013) 4974-4984).

In certain embodiments of all aspects and embodiments, rAAV particle purification is via affinity chromatography, followed by purification via anion exchange chromatography, followed by purification via size exclusion chromatography (SEC).

In certain embodiments of all aspects and embodiments, rAAV particle purification is via affinity chromatography, followed by purification via size exclusion chromatography (SEC), followed by purification via anion exchange chromatography.

Cation exchange chromatography functions to separate the AAV particles from cellular and other components present in the clarified lysate and/or column eluate from an affinity or size exclusion chromatography. Examples of strong cation exchange resins capable of binding rAAV particles over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, MA, USA). Additional examples include Capto S, Capto S ImpAct, Capto S ImpRes (strong cation exchangers available from GE Healthcare, Marlborough, MA, USA), and commercial DOWEX®, AMBERLITE®, and AMBERLYST® families of resins available from Aldrich Chemical Company (Milliwaukee, WI, USA). Weak cation exchange resins include, without limitation, any carboxylic acid based resin. Exemplary cation exchange resins include carboxymethyl (CM), phospho (based on the phosphate functional group), methyl sulfonate(S) and sulfopropyl (SP) resins.

Anion exchange chromatography functions to separate AAV particles from proteins, cellular and other components present in the clarified lysate and/or column eluate from an affinity or cation exchange or size exclusion chromatography. Anion exchange chromatography can also be used to reduce and thereby control the amount of empty capsids in the eluate. For example, the anion exchange column having rAAV particle bound thereto can be washed with a solution comprising NaCl at a modest concentration (e.g., about 100-125 mM, such as 110-115 mM) and a portion of the empty capsids can be eluted in the flow through without substantial elution of the rAAV particles. Subsequently, rAAV particles bound to the anion exchange column can be eluted using a solution comprising NaCl at a higher concentration (e.g., about 130-300 mM NaCl), thereby producing a column eluate with reduced or depleted amounts of empty capsids and proportionally increased amounts of rAAV particles comprising an rAAV vector.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, CA, USA); UNOSPHERE Q (strong anion-exchanger available from BioRad, Hercules, CA, USA); POROS 50HQ (strong anion-exchanger available from Applied Biosystems, Foster City, CA, USA); POROS XQ (strong anion-exchanger available from Applied Biosystems, Foster City, CA, USA); POROS SOD (weak anion-exchanger available from Applied Biosystems, Foster City, CA, USA); POROS 50PI (weak anion-exchanger available from Applied Biosystems, Foster City, CA, USA); Capto Q, Capto XQ, Capto Q ImpRes, and SOURCE 30Q (strong anion-exchanger available from GE healthcare, Marlborough, MA, USA); DEAE SEPHAROSE (weak anion-exchanger available from Amersham Biosciences, Piscataway, NJ, USA); Q SEPHAROSE (strong anion-exchanger available from Amersham Biosciences, Piscataway, NJ, USA). Additional exemplary anion exchange resins include aminoethyl (AE), diethylaminoethyl (DEAE), diethylaminopropyl (DEPE) and quaternary amino ethyl (QAE).

A manufacturing process to purify recombinant AAV particles intended as a product to treat human disease should achieve the following objectives: 1) consistent particle purity, potency and safety; 2) manufacturing process scalability; and 3) acceptable cost of manufacturing.

Exemplary processes for recombinant AAV particle purification are reported in WO 2019/006390.

The below outlined recombinant adeno-associated virus particle (rAAV particle) purification and production methods are scalable up to large scale. For example, to a suspension culture of 5, 10, 10-20, 20-50, 50-100, 100-200, 200-500 or more liters volume. The recombinant adeno-associated virus particle purification and production methods are applicable to a wide variety of AAV serotypes/capsid variants.

In certain embodiments of all aspects and embodiments, the purification of rAAV particles comprises the steps of:
a) harvesting cultivated recombinant AAV particle producing mammalian cells and cell culture supernatant comprising rAAV particles to produce a mammalian cell cultivation broth;
b) optionally concentrating the harvest produced in step (a) to produce a concentrated mammalian cell cultivation broth;
c) lysing the mammalian cells contained in the mammalian cell cultivation broth produced in step (a) or the concentrated mammalian cell cultivation broth produced in step (b) by bringing the broth in contact with an alkyl polyglucoside detergent to produce a mammalian cell cultivation broth lysate;
d) treating the lysate produced in step (c) to reduce contaminating nucleic acid in the lysate thereby producing a nucleic acid reduced lysate;
e) optionally filtering the nucleic acid reduced lysate produced in step (d) to produce a clarified lysate, and optionally diluting the clarified lysate to produce a diluted clarified lysate;
f) subjecting the nucleic acid reduced lysate obtained in step (d), or clarified lysate or diluted clarified lysate produced in step (e) to an AAV affinity column chromatography to produce a column eluate comprising recombinant AAV particles, thereby separating rAAV particles from protein impurities or other production/process related impurities, and optionally concentrating the column eluate to produce a concentrated column eluate;
thereby purifying recombinant AAV particles.

In certain embodiments, steps (a) to (f) are maintained and combined with the following steps:
g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a diluted second column eluate;
h) optionally subjecting the second column eluate or the diluted second column eluate produced in step (g) to an anion exchange chromatography to produce a third column eluate comprising rAAV particles, thereby separating rAAV particles from protein impurities or other production/process related impurities, and optionally diluting the third column eluate to produce a diluted third column eluate; and
i) filtering the second column eluate or the diluted second column eluate produced in step (g), or filtering the third column eluate or the concentrated third column eluate produced in step (h),
and thereby purifying recombinant AAV particles.

In certain embodiments, steps (a) to (f) are maintained and combined with the following steps:
g) subjecting the column eluate or the diluted column eluate produced in step (f) to a cation exchange column chromatography to produce a second column eluate comprising rAAV particles, thereby separating rAAV particles from protein impurities or other production/process related impurities, and optionally diluting the column eluate to produce a diluted second column eluate;
h) subjecting the column eluate or the diluted column eluate produced in step (g) to an anion exchange chromatography to produce a third column eluate comprising rAAV particles, thereby separating rAAV particles from protein impurities or production/process related impurities, and optionally concentrating the third column eluate to produce a concentrated third column eluate,
and thereby purifying recombinant AAV particles.

In certain embodiments, steps (a) to (g) are maintained and combined with the following step:
g) subjecting the column eluate or the diluted column eluate produced in step (f) to an anion exchange chromatography to produce a second column eluate comprising rAAV particles, thereby separating rAAV particles from protein impurities or production/process related impurities, and optionally concentrating the second column eluate to produce a concentrated second column eluate;
h) subjecting the column eluate or the diluted column eluate produced in step (g) to a cation exchange column chromatography to produce a third column eluate comprising rAAV particles, thereby separating rAAV particles from protein impurities or other production/process related impurities, and optionally concentrating the third column eluate to produce a concentrated third column eluate,
and thereby purifying recombinant AAV particles.

In certain embodiments of all aspects and embodiments, concentrating of step (b) and/or step (f) and/or step (g) and/or step (h) is via ultrafiltration/diafiltration, such as by tangential flow filtration (TFF).

In certain embodiments of all aspects and embodiments, concentrating of step (b) reduces the volume of the harvested cells and cell culture supernatant by about 2-20 fold.

In certain embodiments of all aspects and embodiments, concentrating of step (f) and/or step (g) and/or step (h) reduces the volume of the column eluate by about 5-20 fold.

In certain embodiments of all aspects and embodiments, step (d) comprises treating with a nuclease thereby reducing contaminating nucleic acid. Non-limiting examples of a nuclease include benzonase.

In certain embodiments of all aspects and embodiments, filtering of the clarified lysate or the diluted clarified lysate of step (e) is via a filter. Non-limiting examples of filters are those having a pore diameter of between about 0.1 and 10.0 microns, inclusive.

In certain embodiments of all aspects and embodiments, diluting of the clarified lysate of step (e) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, diluting of the second column eluate of step (g) or the third column eluate of step (h) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, the rAAV particles resulting from step (i) are formulated with a surfactant to produce a rAAV particle formulation.

In certain embodiments of all aspects and embodiments, the anion exchange column chromatography of step (g) and/or (h) comprises polyethylene glycol (PEG) modulated column chromatography.

In certain embodiments of all aspects and embodiments, the anion exchange column chromatography of step (g) and/or (h) is washed with a PEG solution prior to elution of the rAAV particles from the column.

In certain embodiments of all aspects and embodiments, the PEG has an average molecular weight in a range of about 1,000 g/mol to 80,000 g/mol, inclusive.

In certain embodiments of all aspects and embodiments, the PEG is at a concentration of about 4% to about 10% (w/v), inclusive.

In certain embodiments of all aspects and embodiments, the anion exchange column of step (g) and/or (h) is washed with an aqueous surfactant solution prior to elution of the rAAV particles from the column.

In certain embodiments of all aspects and embodiments, the cation exchange column of step (g) and/or step (h) is washed with a surfactant solution prior to elution of the rAAV particles from the column.

In certain embodiments of all aspects and embodiments, the PEG solution and/or the surfactant solution comprises an aqueous Tris-HCl/NaCl buffer, an aqueous phosphate/NaCl buffer, or an aqueous acetate/NaCl buffer.

In certain embodiments of all aspects and embodiments, NaCl concentration in the buffer or solution is in a range of between about 20-300 mM NaCl, inclusive, or between about 50-250 mM NaCl, inclusive.

In certain embodiments of all aspects and embodiments, the surfactant comprises a cationic or anionic surfactant.

In certain embodiments of all aspects and embodiments, the surfactant comprises a twelve carbon chained surfactant.

In certain embodiments of all aspects and embodiments, the surfactant comprises Dodecyltrimethylammonium chloride (DTAC) or Sarkosyl.

In certain embodiments of all aspects and embodiments, the rAAV particles are eluted from the anion exchange column of step (f), (g) and/or (h) with an aqueous Tris-HCl/NaCl buffer.

In certain embodiments of all aspects and embodiments, the Tris-HCl/NaCl buffer comprises 100-400 mM NaCl, inclusive, optionally at a pH in a range of about pH 7.5 to about pH 9.0, inclusive.

In certain embodiments of all aspects and embodiments, the anion exchange column of step (g) and/or (h) is washed with an aqueous Tris-HCl/NaCl buffer.

In certain embodiments of all aspects and embodiments, the NaCl concentration in the aqueous Tris-HCl/NaCl buffer is in a range of about 75-125 mM, inclusive.

In certain embodiments of all aspects and embodiments, the aqueous Tris-HCl/NaCl buffer has a pH from about pH 7.5 to about pH 9.0, inclusive.

In certain embodiments of all aspects and embodiments, the anion exchange column of step (g) and/or (h) is washed one or more times to reduce the amount of empty capsids in the second or third column eluate.

In certain embodiments of all aspects and embodiments, the anion exchange column wash removes empty capsids from the column prior to rAAV particle elution and/or instead of rAAV particle elution, thereby reducing the amount of empty capsids in the second or third column eluate.

In certain embodiments of all aspects and embodiments, the anion exchange column wash removes at least about 50% of the total empty capsids from the column prior to rAAV particle elution and/or instead of rAAV particle elution, thereby reducing the amount of empty capsids in the second or third column eluate by about 50%.

In certain embodiments of all aspects and embodiments, the NaCl concentration in the aqueous Tris-HCl/NaCl buffer is in a range of about 110-120 mM, inclusive.

In certain embodiments of all aspects and embodiments, ratios and/or amounts of the rAAV particles and empty capsids eluted are controlled by a wash buffer.

In certain embodiments of all aspects and embodiments, the rAAV particles are eluted from the cation exchange column of step (g) or/and (h) in an aqueous phosphate/NaCl buffer, or an aqueous acetate/NaCl buffer. Non-limiting NaCl concentration in a buffer is in a range of about 125-500 mM NaCl, inclusive. Non-limiting examples of buffer pH are between about pH 5.5 to about pH 7.5, inclusive.

In certain embodiments of all aspects and embodiments, the anion exchange column of step (g) and/or (h) comprises a quaternary ammonium functional group such as quaternized polyethylenimine.

In certain embodiments of all aspects and embodiments, the size exclusion column (SEC) has a separation/fractionation range (molecular weight) from about 10,000 g/mol to about 600,000 g/mol, inclusive.

In certain embodiments of all aspects and embodiments, the cation exchange column of step (g) or/and (h) comprises a sulfonic acid or functional group such as sulphopropyl.

In certain embodiments of all aspects and embodiments, the AAV affinity column comprises a protein or ligand that binds to AAV capsid protein. Non-limiting examples of a protein include an antibody that binds to AAV capsid protein. More specific non-limiting examples include a single-chain Llama antibody (Camelid) that binds to AAV capsid protein.

In certain embodiments of all aspects and embodiments, the method excludes a step of cesium chloride gradient ultracentrifugation.

In certain embodiments of all aspects and embodiments, the method produces rAAV particles having a greater purity than rAAV particles produced or purified by a single AAV affinity column purification.

In certain embodiments of all aspects and embodiments, steps (c) and (d) are performed substantially concurrently.

In certain embodiments of all aspects and embodiments, the NaCl concentration is adjusted to be in a range of about 100-400 mM NaCl, inclusive, or in a range of about 140-300 mM NaCl, inclusive, after step (c) but prior to step (f).

In certain embodiments of all aspects and embodiments, the cells are suspension growing or adherent growing cells.

In certain embodiments of all aspects and embodiments, the cells are mammalian cells. Non-limiting examples include HEK cells, such as HEK-293 cells, and CHO cells, such as CHO-K1 cells.

Methods to determine infectious titer of rAAV particles containing a transgene are known in the art (see, e.g., Zhen et al., Hum. Gene Ther. 15 (2004) 709). Methods for assaying for empty capsids and rAAV particles with packaged transgenes are known (see, e.g., Grimm et al., Gene Therapy 6 (1999) 1322-1330; Sommer et al., Malec. Ther. 7 (2003) 122-128).

To determine the presence or amount of degraded/denatured capsid, purified rAAV particle can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

### DESCRIPTION OF SPECIFIC EMBODIMENTS OF THE INVENTION

The current invention is based at least in part on the finding that the recovery, i.e. yield, of recombinant AAV particles (both capsid-based yield as well as genome-based yield) in an AAV affinity chromatography is influenced/is depending on the detergent used for lysing the cells that produced the recombinant AAV particle prior to the AAV affinity chromatography.

The current invention is further based at least in part on the finding that the ratio of full recombinant AAV particles to empty recombinant AAV particles, which are obtained in an AAV affinity chromatography, is influenced/is depending on the detergent used for lysing the cells that produced the recombinant AAV particle prior to the AAV affinity chromatography.

It has been found that both (capsid-based as well as genomic) yield as well as the ratio of full to empty recombinant AAV particles can be increased in an AAV affinity chromatography by using an alkyl polyglucoside detergent for lysing the cells that produced the recombinant AAV particle prior to the AAV affinity chromatography step.

The method according to the invention is in the following exemplified using Triton CG 110 as an example of an alkyl polyglucoside detergent. This is presented solely as an exemplification of the inventive concept and shall not be construed as limitation. Likewise, any other alkyl polyglucoside detergent can be used. The true scope of the invention is set forth in the appended claims.

In more detail, an increase in the absolute AAV particle recovery in an AAV affinity chromatography of more than 5% can be achieved by using Triton CG 110 as an example of an alkyl polyglucoside detergent compared to the current “gold-standard” Triton X-100 (p-tert octylphenol derivative with a polyethylene glycol side chain), i.e. an absolute recovery of 76% when Triton CG 110 has been used to lyse the cells compared to an absolute recovery of 72% when Triton X-100 has been used to lyse the cells can be achieved.

As the method is intended for large-scale production of recombinant AAV particles, even a minute relative yield increase in AAV particle recovery results in large absolute yield increase and thereby advantage.

Concomitantly with the increase in absolute AAV particle recovery the ratio of full recombinant AAV particles to empty recombinant AAV particles in the AAV affinity chromatography eluate is increased compared to the ratio in the lysate, which was applied to the AAV affinity chromatography material.

In more detail, it has been found that a genomic recovery increase of more than 60% can be obtained in an AAV affinity chromatography by using Triton CG 110 as an example of an alkyl polyglucoside detergent compared to the current “gold-standard” Triton X-100, i.e. an absolute genomic yield of 4.7×1E11 viral genomes per mL (vg/mL) when Triton CG 110 has been used to lyse the cells compared to an absolute genomic yield of 2.8×1E11 viral genomes per mL (vg/mL) when Triton X-100 has been used to lyse the cells. Also an increase of more than 50% can be obtained for the total viral particle yield in the eluate of an AAV affinity chromatography of a Triton CG 110 lysed cultivation broth compared to a Triton X-100 lysed cultivation broth, i.e. an absolute particle yield of 1.2×1E13 viral particles per mL (vp/mL) when Triton CG 110 has been used to lyse the cells compared to an absolute particle yield of 0.77×1E13 viral particles per mL (vp/mL) when Triton X-100 has been used to lyse the cells.

As the increase in the genomic yield is higher than in the total viral particle yield an increase in the ratio of full recombinant AAV particles to empty recombinant AAV particles is obtained.

The respective data is shown in the following Table:

| | Triton X-100 | Triton CG 110 |
|---|---|---|
| | 1% final | 1% final |
| | concentration | concentration |
| genomic titer lysate [vp/ml] | 1.6 × 10¹¹ | 1.6 × 10¹¹ |
| (adjusted starting titer); same | | |
| volume applied | | |
| capsid titer in Poros AAVX | 0.77 × 10¹³ | 1.2 × 10¹³ |
| eluate [vp/ml] | | |
| genomic titer in Poros AAVX | 2.8 × 10¹¹ | 4.7 × 10¹¹ |
| eluate [vg/ml] | | |
| ratio vg/vp [%] denoting the | 3.6 | 3.9 |
| fraction of encapsidated DNA | | |
| genomic yield after Poros | 72.0 | 76.0 |
| AAVX affinity | | |
| chromatography [%] | | |

Thus, the current invention is based at least in part on the finding that the lysing recombinant AAV particle producing cells using Triton CG 110 as an example of an alkyl polyglucoside detergent results in an increase in the recovery of full recombinant AAV particles.

Different combinations of Triton CG 110 final concentrations and lysis times (incubation time in the presence of Triton CG 110) have been tested in a small scale experiment with aliquots of the same cultivation broth obtained 72 hours post triple PEI-mediated transfection.

It has been found that an incubation time of about 60 min with a final concentration of Triton CG 110 of 1% (v/v) provides for the best overall result. This can be even further improved by the incubation with diatomaceous earth for about 20 min. after the incubation with the alkyl polyglucoside detergent Triton CG 110. These are preferred embodiments of the current invention.

The respective data is shown in the following Table.

| | CG 110 | | diatoma- | | | | | |
|---|---|---|---|---|---|---|---|---|
| | final | | ceous | | | | | |
| | concen- | triton | earth | | turbidity | | | full to |
| | tration | incubation | incubition | turbidity | reduction | vp/mL | vg/mL | empty |
| conditions | [w/v] | time [min.] | time [min.] | [NTU] | [NTU] | [E10] | [E9] | ratio |
| supernatant | | | | 235.00 | | 3.90 | 6.20 | 15.8 |
| before | | | | | | | | |
| lysis | | | | | | | | |
| freeze/thaw | | | | | | 4.10 | 5.40 | 13.0 |
| | 0.1% | 30 | 5 | 195.00 | 40.00 | 6.90 | 5.60 | 8.2 |
| | 0.1% | 120 | 5 | 160.00 | 75.00 | 7.75 | 5.50 | 7.2 |
| | 1.5% | 30 | 5 | 110.00 | 125.00 | 4.40 | 4.75 | 10.8 |
| | 1.5% | 120 | 5 | 85.00 | 150.00 | 5.60 | 5.40 | 9.6 |
| | 0.8% | 75 | 5 | 102.50 | 132.50 | 3.80 | 5.10 | 13.5 |
| | 0.8% | 75 | 5 | 102.50 | 132.50 | 6.20 | 5.50 | 8.8 |
| | 1.0% | 60 | 5 | 105.00 | 130.00 | 4.90 | 5.40 | 11.0 |
| | 1.0% | 60 | 20 | 103.00 | 132.00 | 4.20 | 6.20 | 13.6 |

Mammalian HEK293 cells have been transfected with the required plasmids for recombinant AAV particle production, i.e. a transgene comprising plasmid (transgene plasmid), a plasmid encoding AAV packaging proteins (rep/cap plasmid) and a plasmid comprising the required AAV helper functions not already contained in the HEK293 cells (helper plasmid).

In more detail, for producing recombinant AAV particles pre-cultivated HEK293 cells were cultured in a wave bioreactor (10 L working volume) in a batch process using F17 medium with a start cell density of approximately 1E6 cells/ml.

PEI (Polyethylenimine) mediated transfection with three plasmids (helper plasmid, transgene plasmid, rep/cap plasmid) was performed a few hours after the inoculation of the bioreactor. The total DNA amount was calculated based on the cell density after inoculation (1 μg DNA/E6 cells). The molar ratio of the three plasmids was 1:1:1. The transfection reagent was used with an amount of 2 μg/1 μg plasmid.

The cultivation was performed at a temperature of 37° C., a humidity of 70%, a pCO2 of 5%, at 30-35 rpm stirrer speed without oxygen and pH control. Aeration was done with air at a flow of approximately 500 ml/min. The cultivation was terminated 72 h after transfection.

In an alternative process, the HEK cells can be cultured in a 4-day fed-batch process with a feed and glucose addition with a start cell density of approximately 15×1E5 cells/ml.

PEI (Polyethylenimine) mediated transfection with three plasmids (helper plasmid, transgene plasmid, rep/cap plasmid) triple transfection can be performed 24 h after inoculation. First, 20% v/v fresh media is added to the culture. Subsequently, the transfection mixes are prepared and added to the culture. The total DNA concentration is 3 μg/mL and the molar ratio of the three plasmids is 1:1:1. The transfection reagent is used with a concentration of 2.5 μg/1 μg plasmid and free PEI is added at a concentration of 1.5 μg/ml culture volume.

The feed, e.g. HEK FS feed supplement from Xell AG, is added on day two of the fermentation (24 h post transfection) as a bolus feed. Glucose is added on day one and day three of fermentation as a bolus feed. The pH value is adjusted by addition of CO₂ and 1 M Na₂CO₃, respectively, within 0.05 pH units. Antifoam solution is added if necessary. During the process, the parameters temperature, pH value, and pO₂ are monitored and controlled. The fermentation process is stopped on day 4 (72 h after triple transfection) by the addition of lysis buffer.

All references mentioned herein are incorporated herewith by reference.

The following examples are provided to aid the understanding of the present invention, the true scope of which is set forth in the appended claims. It is understood that modifications can be made in the procedures set forth without departing from the spirit of the invention.

### EXAMPLES

### Materials

### Cell Lines

Commercially available HEK293 cells were used for producing AAV particles using transient transfection with three plasmids.

### Cultivation Materials

Cultivation media were prepared according to the operating instructions of the supplier (HEK ViP NB powder media, Xell AG). HEK FS Feed (Xell AG) and F17 media were bought ready to use. Media and feeds were stored at 4° C. in the dark and consumed according to the manufacturer's instructions. Correction agents were stored at room temperature (glucose solution; sodium carbonate solution; defoamer solution).

### Example 1

### Cultivation of HEK293 Cells and Production of Recombinant AAV Particles

Generally, the cultivation methods have been adapted from standard protocols (see, e.g., Lindl, T., “Zell-und Gewebekultur: Einführung in die Grundlagen sowie ausgewählte Methoden und Anwendungen”, Spektrum Akademischer Verlag GmbH, Heidelberg/Berlin, 2002) and operating instructions of the respective supplier.

### Pre-Cultivation

HEK cells were thawed and propagated in shake flasks at 37° C., 70% humidity, a pCO2 of 5% and a shaking frequency of 120 rpm for two to three weeks in cultivation medium. Cells were split every three to four days and expanded in medium to the volume required for inoculation of the production cultivation.

### Production Cultivation

For producing recombinant AAV particles the pre-cultivated HEK293 cells were cultured in a wave bioreactor (10 L working volume) in a batch process using F17 medium with a start cell density of 10×E5 cells/ml.

PEI (Polyethylenimine) mediated transfection with three plasmids (helper plasmid, transgene plasmid, rep/cap plasmid) was performed a few hours after the inoculation of the bioreactor. The total DNA amount was calculated based on the cell density after inoculation (1 μg DNA/E6 cells). The molar ratio of the three plasmids was 1:1:1. Transfection reagent was PEIpro (Polyplus) with an amount of 2 μg/1 μg plasmid DNA.

The cultivation was performed at a temperature of 37° C., a humidity of 70%, a pCO2 of 5%, at 30-35 rpm stirrer speed without oxygen and pH control. Aeration was done with air at a flow of approximately 500 ml/min. The cultivation was terminated 72 h after transfection.

### Example 2

### Lysis

To release the AAV particles into the cell culture broth, 10% (v/v) of lysis buffer (500 mM TRIS, 20 mM MgCl2, pH 7.5) containing different concentrations of tested detergents was added to the culture broth. Additionally, 50 U/ml DNase I (bovine pancreas, Roche) and 37.5 mM MgSO4 were added. The cell culture broth was then incubated for different times at 37° C. with stirring, without aeration and pH control. After the respective incubation, the lysate was sterile filtered.

### Example 3

### AAV Particle Purification

For the affinity chromatography step a column comprising 10.5 mL AAVX resin from Thermo Fisher was used on an Äkta Avant 25 chromatography system. The system was run at a flow rate of about 300 cm/h. After equilibration with buffer A (1×PBS, pH 7.4, 0.001% Pluronic F-68) 200 mL of the lysed culture broth was applied to the column followed by 2 wash steps with equilibration buffer and 0.5 M NaCl, pH 6.0, respectively. AAV particles were eluted with 0.1 M sodium citrate solution, pH 2.4. The pH of the eluate was adjusted to pH 7.5 by addition of 2 M Tris, pH 10.

| | | column |
|---|---|---|
| step | buffer | volumes [CV] |
| equilibration | 1xPBS, pH 7.4 | 3 |
| load | lysate | |
| wash I | 1xPBS, pH 7.4 | 4 |
| wash II | 0.5M sodium chloride pH 6.0 | 4 |
| wash III | 1xPBS, pH 7.4 | 4 |
| elution | 0.1M sodium citrate pH 2.4 | 3 |

### Example 4

### Analytical Methods

### Enzyme-Linked Immunosorbent Assay (ELISA) for Total Titer Determination

For AAV capsid titer determination the kit from PROGEN (Cat. no PrAAV8) was used according to the manufacturer's instructions.

In short, this assay is a sandwich ELISA using as capture antibody a recombinant AAV capsid specific antibody and a biotin-labeled detection antibody.

The wells of the pre-coated multi-titer plate (MTP) were incubated overnight with 100 μL standard, sample or control, respectively, at 4° C. The next day the wells were washed three times with ASSB buffer (1×) as provided in the kit. Thereafter 100 μL per well of a solution comprising the biotinylated detection antibody (diluted according to the manufacturer's instructions) were added and incubated for two hours at room temperature with shaking. Afterwards, the wells were washed three times with ASSB buffer (1×) as provided in the kit. In the next step 100 μl of a solution comprising horseradish peroxidase conjugated to streptavidin was added to each well and incubated for 30 min. At room temperature with shaking. Afterwards, the wells were washed three times with ASSB buffer (1×) as provided in the kit. For color reaction 100 μL of a solution comprising ABTS prepared according to the manufacturer's instructions was added to each well and incubated with shaking. The color intensity was determined suing an MTP-ELISA-Reader Versa Max (Molecular Devices) at 405 nm with a reference wavelength of 490 nm until the difference in the extinction between blank and the standard with the highest concentration reaches about 1.5.

Each sample, standard and control was measured in duplicate.

The amount of capsids (capsids/mL) was calculated based on a standard curve determined by a 4-parameter fitting, e.g. according to the WiemerRodbard algorithm, using the average values of the standards.

Digital Droplet Polymerase Chain Reaction (ddPCR) for Genomic Titer Determination

### Reagents for Enzymatic Sample Treatment:

1) DNase I buffer (NEB): 100 mM Tris-HCl, pH 7.6, 25 mM MgSO4, 5 mM CaCl2
2) DNase I (NEB): 0.2 U/μL
3) Proteinase K (NEB; approx. 20 mg/mL=800 U/mL): 16 U/mL
4) proteinase K buffer (BioRad): 400 mM Tris-HCl, 20 mM EDTA, 2000 mM NaCl, 1% SDS, pH 8
5) sodium dodecyl sulfate (SDS) solution: 10% (w/v)

### Enzymatic Sample Treatment:

mix 30 μL H2O, 5 μL DNase I buffer, 5 μL DNase I, 10 μL sample
incubate at 37° C. for 30 min.
heat to 75° C. for 15 min. to obtain an incubated DNase I-Mix
short cool down and centrifugation
mix 42 μL H2O+2 μL proteinase K+5 μL proteinase K buffer+1 μL 10% SDS solution and add the incubated DNase I-Mix
incubate for 60 min. at 50° C.
heat to 95° C. for 15 min.
cooling to 4° C.
   ddPCR:

For viral genome titration, a duplexing ddPCR assay was performed. Primer and probes were designed against the used CMV promoter and against the polyA/3′UTR sequence. The PCR mastermix was prepared according to the following Table (droplet digital PCR guide-Bio-Rad).

| | volume per | final |
|---|---|---|
| components | well [μL] | concentration |
| Supermix (2x) | 11 | 1x |
| 20 μM CMV primer fwd. | 0.99 | 900 nM |
| 20 μM CMV primer rev | 0.99 | 900 nM |
| 20 μM CMV probe | 0.275 | 250 nM |
| template | 5.5 | — |
| water | 0.99 | — |
| total | 22 | |

The prepared mastermix was pipetted into a 96 well plate with 16.5 μL per well. Then, dilution series of the pretreated samples were conducted: 10 μL of samples were transferred with LoRentention Tips into 90 μL water in LoBind Tubes and thoroughly mixed. Thereafter, 5.5 μL of the samples were added to the mastermix solution in the 96 well plate in several dilution steps. The plate was sealed at 180° C., vortexed at 2,200 rpm for 1 min. and centrifuged at 1,000 rpm for another 1 min. With an automatic droplet generator device, which takes 20 μL PCR mix out of each well, up 20,000 droplets per well were produced and transferred into another 96 well plate. After sealing the droplet plate at 180° C., a PCR run was carried out. The respective conditions are shown in the following Table.

| number of | | | final | |
|---|---|---|---|---|
| cycles | denaturation | annealing | elongation | end |
| 1 | 94° C., 10 min. | | | |
| 39 | 94° C., 30 sec. | 58° C., 1 min. | | |
| 1 | | | 98° C., | 12° C., |
| | | | 10 min. | forever |

In a droplet reader, the fluorescence signal was measured for each droplet. The QuantaSoft software processed the reader data and calculated copy numbers per 20 μL well for the target sequences. Initial sample titers can be determined with following equation:

$$copy number [ \frac{copies}{mL} ] = \frac{output [ \frac{copies}{20 μL well} ]}{5 [ \frac{μL sample}{20 μL well} ]} \cdot dilution factor \cdot 1000 [ \frac{μL}{mL} ]$$

1. A method for purifying recombinant AAV particles in mammalian cell cultivation broth comprising mammalian cells producing said recombinant AAV particles, comprising the following steps:
releasing recombinant AAV particles from said mammalian cells by contacting the mammalian cell cultivation broth with an alkyl polyglucoside detergent, and
purifying the recombinant AAV particles with an AAV affinity chromatography thereby purifying the recombinant AAV particles,
wherein the mammalian cell cultivation broth comprises cultivation medium used for the cultivation of said recombinant AAV particle producing mammalian cells.

2. method of claim 1, wherein the yield is determined after a subsequent AAV affinity chromatography step.

3. The method of claim 1, wherein the recombinant AAV particle producing mammalian cells are lysed with the alkyl polyglucoside detergent prior to the AAV affinity chromatography.

4. The method and use according to claim 1, wherein the alkyl polyglucoside detergent is a mixture of 58.0% to 62.0% (w/v) D-glucopyranose, oligomeric, decyl octyl glycoside and 38.0% to 42.0% (w/v) water.

5. The method and use according to claim 1, wherein the alkyl polyglucoside detergent has the CAS number 68515-73-1.

6. The method and use according to claim 1, wherein the alkyl polyglucoside detergent is in a solution.

7. The method and use according to claim 1, wherein the mammalian cell cultivation broth is combined with 5% to 10% (v/v) with a solution comprising an alkyl polyglucoside detergent.

8. The method and use according to claim 6, wherein the solution comprising the alkyl polyglucoside detergent comprises the alkyl polyglucoside detergent at a concentration of about 10%.

9. The method according to claim 1, wherein bringing into contact or the contacting, respectively, with the alkyl polyglucoside detergent is for about 60 min.

10. The method according to claim 1, wherein said contacting is at a temperature of about 37° C.

11. The method according to claim 1, wherein after said contacting, detergent diatomaceous earth is added to said cell cultivation broth and said cell cultivation broth is incubated for 5 to 20 minutes.

12. The method according to claim 6, wherein the solution comprising the alkyl polyglucoside detergent further comprises magnesium (II) chloride at a concentration of 10 mM to 40 mM.

13. The method according to claim 6, wherein the solution comprising the alkyl polyglucoside detergent has a pH value of about 7.5.

14. The method according to claim 1, wherein the mammalian cell cultivation broth is additionally brought in contact with 50 U/mL of a nuclease selected from DNase I and Benzonase.

15. The method according to claim 1, wherein the affinity chromatography is on a chromatography material comprising a crosslinked poly(styrene-divinyl benzene) matrix to which a single-domain antibody fragment is covalently conjugated, wherein the single-domain antibody fragment specifically binds to AAV serotypes AAV1, AAV2, AAV3, AAV4, AAV5, AAV6, AAV7, AAV8, AAVrh10 and synthetic serotypes based thereon.
